(19) United States
(12) Patent Application Publication
(10) Pub. No.: US 2026/0207616 A1
(43) Pub. Date: Jul. 23, 2026

(54) COMBINATION THERAPIES FOR TREATMENT OF HEARING DISORDERS

(71) Applicant: GATEWAY BIOTECHNOLOGY, INC., St. Louis, MO (US)

(72) Inventor: Jianxin BAO, St. Louis, MO (US)

(21) Appl. No.: 19/138,957

(22) PCT Filed: Jan. 5, 2024
(86) PCT No.: PCT/US2024/010520

§ 371 Date: Jun. 13, 2025

Related U.S. Application Data
(60) Provisional application No. 63/478,889, filed on Jan. 6, 2023.

(51) Int. Cl. A61K 31/55; A61K 31/4422; A61K 31/4748; A61P 27/16

(52) U.S. Cl. CPC A61K 31/55; A61K 31/4422; A61K 31/4748; A61P 27/16

(57) ABSTRACT

Provided herein are methods and compositions for the treatment of hearing disorders by administration of a combination of two or more calcium channel blockers.

### CROSS REFERENCE

This application claims the benefit of U.S. Provisional Application No. 63/478,889, filed on Jan. 6, 2023, which is incorporated herein by reference in its entirety.

### BACKGROUND

Hearing disorders are a growing health problem, with varied and complex etiology. While some forms of hearing disorders are clearly genetic in origin, others are either wholly or at least partially environmental in nature. One such common hearing disorder is tinnitus; a ringing of the ears that may lead to fatigue, stress, trouble sleeping, trouble concentrating, memory problems, depression, anxiety, irritability, and headaches, among other problems, making it a debilitating condition. Tinnitus affects about 20% of the population, and is especially common in older adults, or in people who are routinely exposed to loud sounds as an occupational hazard.

Meniere's disease is a disorder of the inner ear that can lead to ringing in the ears (tinnitus), reduction or loss in hearing, vertigo and a feeling of fullness or congestion in the ear. Attacks of dizziness may come on suddenly or after a short period of tinnitus or muffled hearing. Some people will have single attacks of dizziness separated by long periods of time. Others may experience many attacks closer together over a number of days. Some people with Meniere's disease have vertigo so extreme that they lose their balance and fall. Meniere's disease is often a severe and debilitating condition for afflicted patients.

There are very few options for the treatment of hearing disorders, such as tinnitus and Meniere's disease, and most available treatments are non-pharmaceutical based treatments which do not directly treat the constant ringing of the ears but are directed towards the patient's subjective management of the condition, for example, white noise machines, or counseling. There remains a need in the art for pharmaceutical compositions for reducing, ameliorating, and/or counteracting one or more symptoms of a hearing disorder.

### SUMMARY

Disclosed herein are combinations of pharmaceutical compositions for the treatment of hearing disorders, for example, tinnitus, and Meniere's disease. The present disclosure relates to a novel combination of therapeutic agents for the treatment of hearing disorders, which display a superior effect over the individual agents acting alone.

In one aspect, described herein are methods for treating a hearing disorder or a symptom of a hearing disorder in a subject in need thereof. In some embodiments, the method comprises administering a therapeutically effective amount of a pharmaceutical composition comprising: a first agent comprising a first calcium channel blocker, and a second agent comprising a second calcium channel blocker. In some embodiments, the first agent is nimodipine (NMDP) or a salt thereof and the second agent is carbamazepine (CBZ) or a salt thereof. In some embodiments, the first agent is amlodipine (AML) or a salt thereof and the second agent is carbamazepine or a salt thereof. In some embodiments, the first agent is nimodipine or a salt thereof and the second agent is amlodipine or a salt thereof. In some embodiments, the first agent is tetrandrine (TET) or a salt thereof and the second agent is nimodipine or a salt thereof. In some embodiments, the first agent is tetrandrine or a salt thereof and the second agent is carbamazepine or a salt thereof. In some embodiments, the first agent is tetrandrine or a salt thereof and the second agent is amlodipine or a salt thereof.. In some embodiments, the first agent is an L-type calcium channel blocker. In some embodiments the L-type calcium channel blocker is amlodipine. In some embodiments, the L-type calcium channel blocker is nimodipine. In some embodiments, the second agent is a potassium channel opener (e.g. retigabine). In some embodiments, the second agent is a GABAA receptor positive allosteric modulator (e.g. zolpidem). In some embodiments, the second agent is a dopamine receptor antagonist (e.g. risperidone). In some embodiments, the second agent is a serotonin antagonist and reuptake inhibitor (SARI, e.g. trazadone or nefazodone). In some embodiments the second agent is a sodium channel blocker (e.g. carbamazepine). In some embodiments, wherein the second agent comprises an SARI or a dopamine receptor antagonist the methods can further treat anxiety, insomnia, or depression. In some embodiments, the first agent and the second agent are administered sequentially. In some embodiments, the first agent and the second agent are administered simultaneously.

In another aspect, disclosed herein are methods of selecting a subject in need thereof for treatment of a hearing disorder or a symptom of a hearing disorder in the subject. In some embodiments, the method comprises administering to the subject a test agent comprising an L-type calcium channel blocker or an N-type calcium channel blocker. In some embodiments, the method comprises assessing if the subject was responsive to the test agent. In some embodiments, the method comprises selecting the subject for administration of a first pharmaceutical composition if the subject was responsive to the test agent comprising an L-type calcium channel blocker, or selecting the subject for administration of a second pharmaceutical composition if the subject was responsive to the test agent comprising an N-type calcium channel blocker.

In some embodiments, the method comprises administering a therapeutically effective amount of the first pharmaceutical composition, or a therapeutically effective amount of the second pharmaceutical composition. In some embodiments, the first pharmaceutical composition comprises: i) a first agent comprising a first L-type calcium channel blocker, and ii) a second agent comprising a second N-type calcium channel blocker, a T-type calcium channel blocker, an L-type calcium channel blocker, or a non-specific calcium channel blocker. In some embodiments, the second pharmaceutical composition comprises i) a third agent comprising a first N-type calcium channel blocker, and ii) a fourth agent comprising a second N-type calcium blocker, or an L-type calcium channel blocker. In some embodiments, the test agent is selected from a group consisting of tetrandrine or a salt thereof, nimodipine or a salt thereof, carbamazepine or a salt thereof, and amlodipine or a salt thereof. In some embodiments, the first agent is selected from a group consisting of tetrandrine or a salt thereof, nimodipine or a salt thereof, and amlodipine or a salt thereof. In some embodiments, the first agent comprising the L-type calcium channel blocker comprises NMDP, CBZ, or AML. In some embodiments, the second agent comprising a second N-type calcium channel blocker, a T-type calcium channel blocker, an L-type calcium channel blocker, or a non-specific calcium channel blocker comprises TET.

In some embodiments, the second agent is selected from a group consisting of tetrandrine or a salt thereof, nimodipine or a salt thereof, amlodipine or a salt thereof, and carbamazepine or a salt thereof. In some embodiments, the third agent is selected from a group consisting of tetrandrine or a salt thereof and carbamazepine or a salt thereof. In some embodiments, the fourth agent is selected from a group consisting of tetrandrine or a salt thereof, nimodipine or a salt thereof, amlodipine or a salt thereof, and carbamazepine or a salt thereof. In some embodiments, the method further comprising administering the pharmaceutical composition orally, intratympanically, buccally, intradermally, transdermally, topically, or via inhalation.

In some embodiments, the method comprises administering the pharmaceutical composition intravenously to the subject in need thereof. In some embodiments, the method comprises orally administering the pharmaceutical composition to the subject in need thereof. In some embodiments, the method comprises administering the pharmaceutical composition via intratympanic injection to the subject in need thereof. In some embodiments, the method comprises administering the pharmaceutical composition by bringing the composition in contact with the crista fenestrae cochlea, the round window, the tympanic cavity, the tympanic membrane, the auris media or the auris externa.

In some embodiments, the method comprises administering the pharmaceutical composition into the ear canal, or in the vestibule of the ear. In some embodiments, the method comprises administering the pharmaceutical composition via a single dose intratympanic injection. In some embodiments, the method comprises administering the pharmaceutical composition at any time before or after onset of symptoms of the hearing disorder which may be treatable with the pharmaceutical composition. In some embodiments, the method comprises administering the pharmaceutical composition about every 4 hours. In some embodiments, the method comprises administering the pharmaceutical composition about every 2 hours.

In some embodiments, the method comprises administering the pharmaceutical compositions about every 8 hours. In some embodiments, the method comprises administering the pharmaceutical composition at least once a day, twice a day, three times a day, four times a day, five times a day, six times a day, seven times a day, eight times a day, nine times a day, or ten times a day.

In some embodiments, the method comprises administering the pharmaceutical composition weekly. In some embodiments, the method comprises administering the pharmaceutical composition via a low-dose therapy. In some embodiments, the method comprises administering the pharmaceutical composition continuously. In some embodiments, the method comprises administering the pharmaceutical composition by titration to vestibular symptoms. In some embodiments, the method comprises administering the pharmaceutical composition for at least one day, at least two days, at least three days, at least four days, at least five days, at least six days, at least one week, at least two weeks, at least three weeks, at least four weeks, at least one month, at least two months, at least six months, or at least one year. In some embodiments, the method comprises administering to the subject another active pharmaceutical composition or salt thereof. In some embodiments, the method comprises administering the pharmaceutical composition in a unit dosage form.

In some embodiments, the method comprises administering the pharmaceutical composition in order to not cause a change in cochlear potential. In some embodiments, the method comprises increasing the subject's dose of the pharmaceutical composition until a symptom of inner ear disturbance in the subject is observed. In some embodiments, the method comprises increasing the subject's dose of the pharmaceutical composition until the patient reports no instances of vertigo, or reduced instances of vertigo. In some embodiments, the method comprises administering the pharmaceutical composition via a drug delivery device. In some embodiments, the method comprises delivering the pharmaceutical composition via a dropper bottle, needle, syringe, pump, microinjection device, microcatheter, or combinations thereof. In some embodiments, the method comprises ceasing administration of the pharmaceutical composition upon observing a symptom of inner ear disturbance in the subject.

In some embodiments, the symptom of inner ear disturbance comprises spontaneous nystagmus observed with Frenzel's glasses, disequilibrium, motion intolerance, or reduction/loss in hearing. In some embodiments, the pharmaceutical composition is administered in at least about 1 mg/ml. In some embodiments, the pharmaceutical composition is administered in a unit dose of about 1 mg to about 1000 mg. In some embodiments, the pharmaceutical composition is administered in a unit dose of about 1 mg to about 100 mg. In some embodiments, the hearing disorder in the subject in need thereof is noise-induced hearing loss (NIHL). In some embodiments, the hearing disorder in the subject in need thereof is age-related hearing loss (ARHL) or presbycusis. In some embodiments, the hearing disorder in the subject in need thereof is injury-or drug-induced hearing loss.

In some embodiments, the hearing disorder in the subject in need thereof is tinnitus, or Meniere's disease. In some embodiments, the therapeutically effective amount of NMDP is from about 50 mg to about 1000 mg/kg of the subject in need thereof. In some embodiments, the therapeutically effective amount of AML is from about 50 mg to about 1000 mg/kg of the subject in need thereof. In some embodiments, the therapeutically effective amount of CBZ is about 50 mg to about 1000 mg/kg of the subject in need thereof. In some embodiments, the therapeutically effective amount of TET is from about 50 mg to about 1000 mg/kg of the subject in need thereof. In some embodiments, the effect of the test agent on the subject is measured by an assessment selected from the group consisting of a) measurement of a change in ABR threshold, b) a change in auditory speech recognition as measured by a words-in-noise test, c) a change in auditory speech recognition as measured by a digits-in-noise test, d) a change in low frequency hearing thresholds e) a change in incidence of adverse events after administration of the test agent, f) a change in tinnitus severity g) a change in tinnitus loudness h) a change in vertigo severity i) a change in aural fullness j) a change in dizziness, and k) a change in hair cell function as observed when measured by a change in ABR threshold, after administration of the first or second test agent.

In some embodiments, the measurement of the ABR threshold is performed at the frequency range of 5 kHz-50 kHz. In some embodiments, the therapeutically effective amount of the first agent and the therapeutically effective amount of the second agent are less than the amounts of the first agent or the second agent required to treat a hearing disorder when administered individually. In some embodiments, the amounts of the therapeutically effective amount of the third agent and the therapeutically effective amount of the fourth agent provide a synergistic effect. In some embodiments, the therapeutically effective amount of the third agent and the therapeutically effective amount of the fourth agent are less than the amounts of third agent or fourth agent required to treat a hearing disorder when administered individually. In some embodiments, the amounts of the therapeutically effective amount of the third agent and the therapeutically effective amount of the fourth agent provide a synergistic effect.

In another aspect, described herein are pharmaceutical compositions for treating a symptom of a hearing disorder in a subject in need thereof. In some embodiments, the pharmaceutical composition comprises: a pharmaceutically effective amount of tetrandrine (TET) or a salt thereof and a pharmaceutically effective amount of nimodipine or a salt thereof. In some embodiments, the pharmaceutical composition comprises: a pharmaceutically effective amount of tetrandrine or a salt thereof and a pharmaceutically effective amount of amlodipine or a salt thereof. In some embodiments, the pharmaceutical composition comprises: a pharmaceutically effective amount of tetrandrine or a salt thereof and a pharmaceutically effective amount of carbamazepine (CBZ) or a salt thereof. In some embodiments, the pharmaceutical composition comprises: a pharmaceutically effective amount of amlodipine (AML) or a salt thereof and a pharmaceutically effective amount of carbamazepine or a salt thereof. In some embodiments, the pharmaceutical composition comprises: a pharmaceutically effective amount of nimodipine (NMDP) or a salt thereof and a pharmaceutically effective amount of amlodipine or a salt thereof. In some embodiments, the pharmaceutical composition comprises: a pharmaceutically effective amount of nimodipine or a salt thereof and a pharmaceutically effective amount of carbamazepine or a salt thereof.

In some embodiments, the concentration of NMDP in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300,, 350, or 400 mg/ml. In some embodiments, the concentration of TET in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, or 400 mg/ml. In some embodiments, the concentration of CBZ in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300,, 350, or 400 mg/ml.

In some embodiments, the concentration of AML in the pharmaceutical composition administered is 0.5, 1, 1.5, 2, 2.5, 3, 3.5, 4, 4.5, 5, 5.5, 6, 6.5, 7, 7.5, 8, 8.5, 9, 9.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, or 400 mg/ml. In some embodiments, the volume of the pharmaceutical composition administered is 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 ml. In some embodiments, the pharmaceutical composition is released either continuously, variably or in a pulsatile manner, or combinations thereof. In some embodiments, the pharmaceutical composition is in a unit dosage form. In some embodiments, the unit dosage form is in a tablet, capsule, caplet, gel cap, powder, or solution dosage form. In some embodiments, the composition is aqueous.

In some embodiments, the composition is in the form of gel or film. In some embodiments, the composition comprises micronized particles. In some embodiments, the unit dosage form has a unit weight of from about 10 mg to about 10 g. In some embodiments, the pharmaceutical composition comprises NMDP at a concentration of from about 0.1% to about 20% w/w of the formulation. In some embodiments, the pharmaceutical composition comprises tetrandrine or the salt thereof at a concentration of from about 0.1% to about 20% of the formulation. In some embodiments, the pharmaceutical composition comprises carbamazepine or the salt thereof at a concentration of from about 0.1% to about 20% of the formulation. In some embodiments, the pharmaceutical composition comprises amlodipine or the salt thereof at a concentration of from about 0.1% to about 20% of the formulation.

These and other objects and features of the disclosure will be more fully appreciated when the following detailed description of the disclosure is read in conjunction with the accompanying examples and drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows the results of the go/no-go trials for TET administered at dosages of 30, 60, and 90 mg/kg;

FIG. 1B shows the results of the go/no-go trials for NMDP administered at dosages of 10, 30, and 50 mg/kg;

FIG. 1C shows the results of the go/no-go trials for AML administered at dosages of 5, and 8 mg/kg;

FIG. 1D shows the results of the go/no-go trials for CBZ administered at dosages of 5, and 8 mg/kg;

FIG. 2A shows the results of the go/no-go trials for TET and NMDP administered at dosages of 10 mg/kg each;

FIG. 2B shows the results of the go/no-go trials for ESM and GAB administered at 300 mg/kg ESM and 20 mg/kg GAB, and at 300 mg/kg ESM and 40 mg/kg GAB;

FIG. 3 summarizes the go/no-go trial sound based avoidance detection methodology utilized herein.

FIG. 4 shows the results of the no-go trials for TET and NMDP administered at 10+10 mg/kg, CBZ administered at 5 and 8 mg/kg and CBZ and NMDP administered at 5+10 mg/kg.

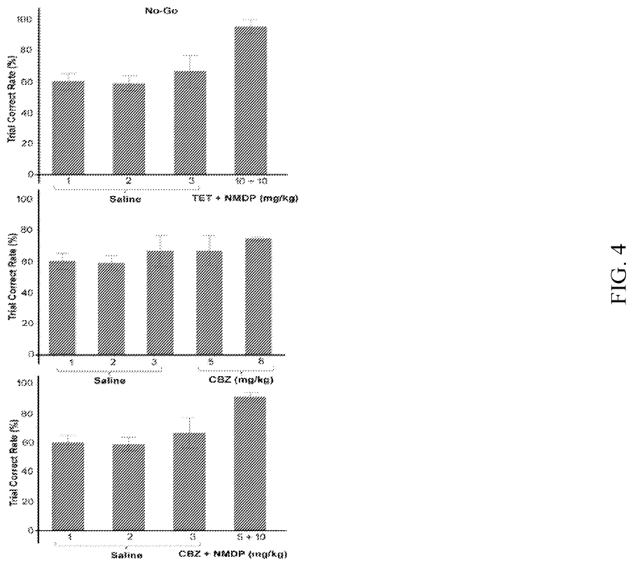

### DETAILED DESCRIPTION

Responsive to the unmet need in the art for pharmaceutical compositions for treating hearing disorders, disclosed herein are pharmaceutical compositions and combination therapies for the treatment of hearing disorders, for example, tinnitus, or Meniere's disease. The present disclosure relates to novel pharmaceutical formulations and treatment methods for delivering an effective dose of a first therapeutic agent or a first therapeutic agent and a second therapeutic agent for the treatment of a hearing disorder (e.g. tinnitus, or Meniere's disease). The first agent can comprise a first calcium channel blocker and the second agent can comprise a second calcium channel blocker. The first agent can comprise a first L-type calcium channel blocker and the second agent can comprise a second L-type calcium channel blocker, a T-type calcium channel blocker, an N-type calcium channel blocker, and/or a non-specific calcium channel blocker, a potassium channel opener, a sodium blocker, a GABAA modulator, and/or one or more SARIs. Co-administration of the first and second agents can be either sequential or simultaneous. Simultaneous administration can comprise administration of a pharmaceutical composition or formulation comprising both the first and second agents as described herein.

Compositions of this disclosure can be administered by any route typical for administering a pharmaceutical formulation, such as intravenous administration, injection, delivery, or oral administration.

The present investigators have developed compositions and methods of treatment comprising use of a first and/or second agent to alleviate the symptoms of tinnitus, or Meniere's disease after the administration of one or more doses of the composition.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “nimodipine” is intended to relate to nimodipine or a pharmaceutically acceptable salt thereof. The term “equivalent to about. of nimodipine” is intended to relate to a specified volume, concentration, or amount of nimodipine free base provided by a volume, concentration, or amount of a salt of nimodipine (NMDP). Thus, the specified amount relates to the amount of nimodipine (NMDP) free base and not the amount of the nimodipine (NMDP) salt, despite the use of the salt in the composition. In an embodiment, the composition, methods and uses of the present disclosure comprise the use of nimodipine (NMDP) citrate.

The term “tetrandrine” is intended to relate to tetrandrine or a pharmaceutically acceptable salt thereof. The term “equivalent to about . . . of tetrandrine” is intended to relate to a specified volume, concentration, or amount of tetrandrine free base provided by a volume, concentration, or amount of a salt of tetrandrine (TET). Thus, the specified amount relates to the amount of tetrandrine (TET) free base and not the amount of the tetrandrine (TET) salt, despite the use of the salt in the composition. In an embodiment, the composition, methods and uses of the present disclosure comprise the use of tetrandrine (TET) citrate.

The term “carbamazepine” is intended to relate to tetrandrine or a pharmaceutically acceptable salt thereof. The term “equivalent to about . . . of carbamazepine” is intended to relate to a specified volume, concentration, or amount of carbamazepine free base provided by a volume, concentration, or amount of a salt of tetrandrine (CBZ). Thus, the specified amount relates to the amount of carbamazepine (CBZ) free base and not the amount of the carbamazepine (CBZ) salt, despite the use of the salt in the composition. In an embodiment, the composition, methods and uses of the present disclosure comprise the use of carbamazepine (CBZ) citrate.

The term “amlodipine” is intended to relate to tetrandrine or a pharmaceutically acceptable salt thereof. The term “equivalent to about . . . of amlodipine” is intended to relate to a specified volume, concentration, or amount of amlodipine free base provided by a volume, concentration, or amount of a salt of amlodipine (AML). Thus, the specified amount relates to the amount of amlodipine (AML) free base and not the amount of the amlodipine (AML) salt, despite the use of the salt in the composition. In an embodiment, the composition, methods and uses of the present disclosure comprise the use of amlodipine (AML) citrate.

The terms “ethosuximide” and “ESM” are intended to relate to ethosuximide or a pharmaceutically acceptable salt thereof.

The terms “Gabapentin” and “GAB” are intended to relate to gabapentin or a pharmaceutically acceptable salt thereof.

The term “zolpidem” is intended to relate to zolpidem or a pharmaceutically acceptable salt thereof.

The term “retigabine” is intended to relate to zolpidem or a pharmaceutically acceptable salt thereof.

The terms “SARI” and “SARIs” generally refer to serotonin antagonist and reuptake inhibitors and pharmaceutically acceptable salts thereof. Examples of an SARI are trazodone, nefazodone, mCPP, hydroxynefazodone, etoperidone, triazoledione, combinations thereof and/or pharmaceutically acceptable salts thereof.

The term “formulated” is intended to relate to the selection of excipients, carriers, vehicles, preservatives, stabilizing agents and so forth in the preparation of medicament using said composition. The term “formulated” is furthermore intended to relate to the selection of the device for delivery of the composition or selection of containment device for administration or storing of the composition.

The term “dosage unit” relates to the composition administered in one administration by one delivery operation. A delivery operation is an operation which delivers a dosage unit. A treatment relates to the administration of the composition during a single episode of tinnitus, or Meniere's disease or a symptom of tinnitus, or Meniere's disease, said episode lasting until alleviation of tinnitus, or Meniere's disease or a symptom of tinnitus, or Meniere's disease.

The term “time-to-onset-of-action” is intended to mean the moment wherein the patient begins to experience tinnitus, or Meniere's disease or a symptom of tinnitus, or Meniere's disease relief, usually as a result of sufficient plasma concentrations of a first agent comprising a first L-type calcium channel blocker in combination with a second agent comprising either a second L-type calcium channel blocker and/or an N-type calcium channel blocker. Sufficient plasma concentrations to achieve all varies amongst patients, amongst patient classes and types and nature of tinnitus, or Meniere's disease or a symptom of tinnitus, or Meniere's disease experienced. The “action” in “time-to-onset-of-action” is tinnitus, or Meniere's disease or a symptom of tinnitus, or Meniere's disease relief.

The term “duration-of-action” relates to the time throughout which tinnitus, or Meniere's disease or a symptom of tinnitus, or Meniere's disease relief is experienced by the patient.

An “extended time period” intends a period of more than about 4 months, preferably more than about 6 months.

The phrase “amount effective to suppress one or more symptoms of tinnitus, or Meniere's disease” refers to a dose of the therapeutic compound, that reduces one or more symptoms of tinnitus, or Meniere's disease are alleviated in a subject after delivery of the pharmaceutical composition.

The terminology used herein is for describing particular embodiments only and is not intended to be limiting of the disclosure. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

As used herein, the term “about” a number refers to that number plus or minus 10% of that number. The term “about” a range refers to that range minus 10% of its lowest value and plus 10% of its greatest value. For example, the term “about” can be immediately understood as within 10%, 9%, 8%, 7%, 6%, 5%, 4%, 3%, 2%, 1%, 0.5%, 0.1%, 0.05%, or 0.01% of the stated value. As an illustration, a numerical range of “about 1 to about 5” should be interpreted to include not only the explicitly recited values of about 1 to about 5, but also include individual values and sub-ranges within the indicated range. This includes, at very least, the degree of expected experimental error, technical error and instrumental error for a given experiment, technique or an instrument used to measure a value.

As used herein, the term “and/or” includes any combinations of one or more of the associated listed items. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

The terms “active agent”, “pharmaceutical active agent”, “active”, “API”, “active pharmaceutical ingredient”, “active substance”, “active molecule”, “active compound” or “drug” are used interchangeably with L-type calcium channel blockers and/or N-type calcium channel blockers and/or salts thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The disaggregating agent comprised in the disclosed composition is an inert carrier, as described in detail below. It is to be noted that “disaggregating agent”, “carrier”, “diluent” and “deagglomerating agent” are used herein interchangeably, and refer to an inert ingredient added to the pharmaceutical composition, comprising the said second type particles.

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraperitoneal injection and the non-injection methods of delivery to a mucosa.

A “subject” in accordance with some embodiments is a subject in which the symptoms and symptoms, the physical examination results and/or the psychological test results are determined and recorded in relation to the condition of the individual (i.e., the disease or disorder condition). As used herein, the disease or disorder is a hearing disorder. As used herein, the disease or disorder is tinnitus, or Meniere's disease. As used herein, “subject” is intended to be a human subject, but is not necessarily limited thereto. The subject can be male or female and can be any race or ethnic group including, but not limited to, Caucasian, African American, African, Asian, Hispanic, Indians and the like. As used herein, a subject is an animal, particularly a mammal such as a dog, cat, cow, goat, horse, sheep, or both, that can be treated according to the methods of the disclosure or screened for veterinary and pharmaceutical or pharmaceutical drug development purposes, Pigs, rodents (e.g., rats and mice), rabbit necks, primates (including non-human primates), and the like. A subject according to some embodiments of the present disclosure includes a patient or a human in need of a therapeutic treatment for a disorder treatable by compositions described herein

### Methods and Compositions

Methods of this disclosure comprise co-administration of a therapeutic dose of a combination of a first therapeutic agent and a second therapeutic agent for the treatment of a hearing disorder (e.g., tinnitus, or Meniere's disease). The first agent can comprise a first L-type calcium channel blocker and the second agent can comprise a second L-type calcium channel blocker, or an N-type calcium channel blocker. Co-administration of the first and second agents can be either sequential or simultaneous. Simultaneous administration can comprise administration of a pharmaceutical composition or formulation comprising both the first and second agents as described herein.

L-type calcium channel blockers can include, but are not limited to nimodipine, tetrandrine, carbamazepine, amlodipine, aranidipine, azelnidipine, barnidipine, benidipine, efonidipine, and/or nicardipine, and pharmaceutically acceptable salts, solvates, and excipients thereof. N-type calcium channel blockers can include, but are not limited to ω-Conotoxins, cadmium, caroverine, cilnidipine, desipramine, gabapentin, levetiracetam, lamotrigine, nicardipine, piracetam, pregabalin, trox-1, and/or ziconotide and pharmaceutically acceptable salts, solvates, and excipients thereof. T-type calcium channel blockers can include, but are not limited to, tetrandrine, amlodipine, nimodipine, amlodipine, aranidipine, azelnidipine, barnidipine, benidipine, efonidipine, and/or nicardipine and pharmaceutically acceptable salts, solvates, and excipients thereof. Non-specific calcium channel blockers can include, but are not limited to, tetrandrine, tetrandrine, amlodipine, nimodipine, amlodipine, aranidipine, azelnidipine, barnidipine, benidipine, efonidipine, and/or nicardipine and pharmaceutically acceptable salts, solvates, and excipients thereof.

Compositions of this disclosure can be administered by any route typical for administering a pharmaceutical formulation, such as intravenous administration, injection, delivery, or oral administration. The first agent and second agent can be administered either sequentially (e.g. in two or more dose forms) or simultaneously (e.g. in a single dose form).

In some embodiments, a pharmaceutical composition is administered in a dose of about 1 mg to about 1000 mg. In some embodiments, a pharmaceutical composition is administered in a dose of about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 1 mg to about 10 mg, about 1 mg to about 20 mg, about 1 mg to about 50 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 2 mg to about 20 mg, about 2 mg to about 50 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 3 mg to about 20 mg, about 3 mg to about 50 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 4 mg to about 20 mg, about 4 mg to about 50 mg, about 5 mg to about 6 mg, about 5 mg to about 7 mg, about 5 mg to about 8 mg, about 5 mg to about 9 mg, about 5 mg to about 10 mg, about 5 mg to about 20 mg, about 5 mg to about 50 mg, about 6 mg to about 7 mg, about 6 mg to about 8 mg, about 6 mg to about 9 mg, about 6 mg to about 10 mg, about 6 mg to about 20 mg, about 6 mg to about 50 mg, about 7 mg to about 8 mg, about 7 mg to about 9 mg, about 7 mg to about 10 mg, about 7 mg to about 20 mg, about 7 mg to about 50 mg, about 8 mg to about 9 mg, about 8 mg to about 10 mg, about 8 mg to about 20 mg, about 8 mg to about 50 mg, about 9 mg to about 10 mg, about 9 mg to about 20 mg, about 9 mg to about 50 mg, about 10 mg to about 20 mg, about 10 mg to about 50 mg, or about 20 mg to about 50 mg. In some embodiments, a pharmaceutical composition is administered in a dose of about 1 mg, about 2 mg, about 3 mg, about 4 mg, about 5 mg, about 6 mg, about 7 mg, about 8 mg, about 9 mg, about 10 mg, about 20 mg, or about 50 mg. In some embodiments, a pharmaceutical composition is administered in a dose of at least about 1 mg, about 2 mg, about 3 mg, about 4 mg, about 5 mg, about 6 mg, about 7 mg, about 8 mg, about 9 mg, about 10 mg, or about 20 mg. In some embodiments, a pharmaceutical composition is administered in a dose of at most about 2 mg, about 3 mg, about 4 mg, about 5 mg, about 6 mg, about 7 mg, about 8 mg, about 9 mg, about 10 mg, about 20 mg, or about 50 mg. In some embodiments, a pharmaceutical composition is administered in a dose of about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 20 mg to about 150 mg, about 20 mg to about 200 mg, about 20 mg to about 250 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 30 mg to about 150 mg, about 30 mg to about 200 mg, about 30 mg to about 250 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 40 mg to about 150 mg, about 40 mg to about 200 mg, about 40 mg to about 250 mg, about 50 mg to about 60 mg, about 50 mg to about 70 mg, about 50 mg to about 80 mg, about 50 mg to about 90 mg, about 50 mg to about 100 mg, about 50 mg to about 150 mg, about 50 mg to about 200 mg, about 50 mg to about 250 mg, about 60 mg to about 70 mg, about 60 mg to about 80 mg, about 60 mg to about 90 mg, about 60 mg to about 100 mg, about 60 mg to about 150 mg, about 60 mg to about 200 mg, about 60 mg to about 250 mg, about 70 mg to about 80 mg, about 70 mg to about 90 mg, about 70 mg to about 100 mg, about 70 mg to about 150 mg, about 70 mg to about 200 mg, about 70 mg to about 250 mg, about 80 mg to about 90 mg, about 80 mg to about 100 mg, about 80 mg to about 150 mg, about 80 mg to about 200 mg, about 80 mg to about 250 mg, about 90 mg to about 100 mg, about 90 mg to about 150 mg, about 90 mg to about 200 mg, about 90 mg to about 250 mg, about 100 mg to about 150 mg, about 100 mg to about 200 mg, about 100 mg to about 250 mg, about 150 mg to about 200 mg, about 150 mg to about 250 mg, or about 200 mg to about 250 mg. In some embodiments, a pharmaceutical composition is administered in a dose of about 20 mg, about 30 mg, about 40 mg, about 50 mg, about 60 mg, about 70 mg, about 80 mg, about 90 mg, about 100 mg, about 150 mg, about 200 mg, or about 250 mg. In some embodiments, a pharmaceutical composition is administered in a dose of at least about 20 mg, about 30 mg, about 40 mg, about 50 mg, about 60 mg, about 70 mg, about 80 mg, about 90 mg, about 100 mg, about 150 mg, or about 200 mg. In some embodiments, a pharmaceutical composition is administered in a dose of at most about 30 mg, about 40 mg, about 50 mg, about 60 mg, about 70 mg, about 80 mg, about 90 mg, about 100 mg, about 150 mg, about 200 mg, or about 250 mg. In some embodiments, a pharmaceutical composition is administered in a dose of about 100 mg to about 200 mg, about 100 mg to about 250 mg, about 100 mg to about 300 mg, about 100 mg to about 350 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1,000 mg, about 200 mg to about 250 mg, about 200 mg to about 300 mg, about 200 mg to about 350 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 250 mg to about 300 mg, about 250 mg to about 350 mg, about 250 mg to about 400 mg, about 250 mg to about 500 mg, about 250 mg to about 600 mg, about 250 mg to about 700 mg, about 250 mg to about 800 mg, about 250 mg to about 900 mg, about 250 mg to about 1,000 mg, about 300 mg to about 350 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 350 mg to about 400 mg, about 350 mg to about 500 mg, about 350 mg to about 600 mg, about 350 mg to about 700 mg, about 350 mg to about 800 mg, about 350 mg to about 900 mg, about 350 mg to about 1,000 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 500 mg to about 600 mg, about 500 mg to about 700 mg, about 500 mg to about 800 mg, about 500 mg to about 900 mg, about 500 mg to about 1,000 mg, about 600 mg to about 700 mg, about 600 mg to about 800 mg, about 600 mg to about 900 mg, about 600 mg to about 1,000 mg, about 700 mg to about 800 mg, about 700 mg to about 900 mg, about 700 mg to about 1,000 mg, about 800 mg to about 900 mg, about 800 mg to about 1,000 mg, or about 900 mg to about 1,000 mg. In some embodiments, a pharmaceutical composition is administered in a dose of about 100 mg, about 200 mg, about 250 mg, about 300 mg, about 350 mg, about 400 mg, about 500 mg, about 600 mg, about 700 mg, about 800 mg, about 900 mg, or about 1,000 mg. In some embodiments, a pharmaceutical composition is administered in a dose of at least about 100 mg, about 200 mg, about 250 mg, about 300 mg, about 350 mg, about 400 mg, about 500 mg, about 600 mg, about 700 mg, about 800 mg, or about 900 mg. In some embodiments, a pharmaceutical composition is administered in a dose of at most about 200 mg, about 250 mg, about 300 mg, about 350 mg, about 400 mg, about 500 mg, about 600 mg, about 700 mg, about 800 mg, about 900 mg, or about 1,000 mg. In some embodiments, a pharmaceutical composition is administered in a dose of about 1 mg to about 2,000 mg. In some embodiments, a pharmaceutical composition is administered in a dose of about 1 mg to about 50 mg, about 1 mg to about 100 mg, about 1 mg to about 200 mg, about 1 mg to about 300 mg, about 1 mg to about 500 mg, about 1 mg to about 750 mg, about 1 mg to about 1,000 mg, about 1 mg to about 1,250 mg, about 1 mg to about 1,500 mg, about 1 mg to about 1,750 mg, about 1 mg to about 2,000 mg, about 50 mg to about 100 mg, about 50 mg to about 200 mg, about 50 mg to about 300 mg, about 50 mg to about 500 mg, about 50 mg to about 750 mg, about 50 mg to about 1,000 mg, about 50 mg to about 1,250 mg, about 50 mg to about 1,500 mg, about 50 mg to about 1,750 mg, about 50 mg to about 2,000 mg, about 100 mg to about 200 mg, about 100 mg to about 300 mg, about 100 mg to about 500 mg, about 100 mg to about 750 mg, about 100 mg to about 1,000 mg, about 100 mg to about 1,250 mg, about 100 mg to about 1,500 mg, about 100 mg to about 1,750 mg, about 100 mg to about 2,000 mg, about 200 mg to about 300 mg, about 200 mg to about 500 mg, about 200 mg to about 750 mg, about 200 mg to about 1,000 mg, about 200 mg to about 1,250 mg, about 200 mg to about 1,500 mg, about 200 mg to about 1,750 mg, about 200 mg to about 2,000 mg, about 300 mg to about 500 mg, about 300 mg to about 750 mg, about 300 mg to about 1,000 mg, about 300 mg to about 1,250 mg, about 300 mg to about 1,500 mg, about 300 mg to about 1,750 mg, about 300 mg to about 2,000 mg, about 500 mg to about 750 mg, about 500 mg to about 1,000 mg, about 500 mg to about 1,250 mg, about 500 mg to about 1,500 mg, about 500 mg to about 1,750 mg, about 500 mg to about 2,000 mg, about 750 mg to about 1,000 mg, about 750 mg to about 1,250 mg, about 750 mg to about 1,500 mg, about 750 mg to about 1,750 mg, about 750 mg to about 2,000 mg, about 1,000 mg to about 1,250 mg, about 1,000 mg to about 1,500 mg, about 1,000 mg to about 1,750 mg, about 1,000 mg to about 2,000 mg, about 1,250 mg to about 1,500 mg, about 1,250 mg to about 1,750 mg, about 1,250 mg to about 2,000 mg, about 1,500 mg to about 1,750 mg, about 1,500 mg to about 2,000 mg, or about 1,750 mg to about 2,000 mg, including increments therein. In some embodiments, a pharmaceutical composition is administered in a dose of about about 1 mg, about 50 mg, about 100 mg, about 200 mg, about 300 mg, about 500 mg, about 750 mg, about 1,000 mg, about 1,250 mg, about 1,500 mg, about 1,750 mg, or about 2,000 mg. In some embodiments, a pharmaceutical composition is administered in a dose of about at least about 1 mg, about 50 mg, about 100 mg, about 200 mg, about 300 mg, about 500 mg, about 750 mg, about 1,000 mg, about 1,250 mg, about 1,500 mg, or about 1,750 mg. In some embodiments, a pharmaceutical composition is administered in a dose of about at most about 50 mg, about 100 mg, about 200 mg, about 300 mg, about 500 mg, about 750 mg, about 1,000 mg, about 1,250 mg, about 1,500 mg, about 1,750 mg, or about 2,000 mg. In some embodiments, the dose of the pharmaceutical composition is a therapeutically effective dose.

The therapeutically effective amount of a first and/or second can be dependent on the weight of a subject. In some cases, the therapeutically effective amount of a first and/or second is at least about 1 mg of a first and/or second per kg of the subject, for example at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg of a first and/or second per kg of the subject.

In some embodiments, a pharmaceutical composition is administered in about 1 doses/day to about 12 doses/day. In some embodiments, a pharmaceutical composition is administered in about 1 doses/day to about 2 doses/day, about 1 doses/day to about 3 doses/day, about 1 doses/day to about 4 doses/day, about 1 doses/day to about 5 doses/day, about 1 doses/day to about 6 doses/day, about 1 doses/day to about 7 doses/day, about 1 doses/day to about 8 doses/day, about 1 doses/day to about 9 doses/day, about 1 doses/day to about 10 doses/day, about 1 doses/day to about 11 doses/day, about 1 doses/day to about 12 doses/day, about 2 doses/day to about 3 doses/day, about 2 doses/day to about 4 doses/day, about 2 doses/day to about 5 doses/day, about 2 doses/day to about 6 doses/day, about 2 doses/day to about 7 doses/day, about 2 doses/day to about 8 doses/day, about 2 doses/day to about 9 doses/day, about 2 doses/day to about 10 doses/day, about 2 doses/day to about 11 doses/day, about 2 doses/day to about 12 doses/day, about 3 doses/day to about 4 doses/day, about 3 doses/day to about 5 doses/day, about 3 doses/day to about 6 doses/day, about 3 doses/day to about 7 doses/day, about 3 doses/day to about 8 doses/day, about 3 doses/day to about 9 doses/day, about 3 doses/day to about 10 doses/day, about 3 doses/day to about 11 doses/day, about 3 doses/day to about 12 doses/day, about 4 doses/day to about 5 doses/day, about 4 doses/day to about 6 doses/day, about 4 doses/day to about 7 doses/day, about 4 doses/day to about 8 doses/day, about 4 doses/day to about 9 doses/day, about 4 doses/day to about 10 doses/day, about 4 doses/day to about 11 doses/day, about 4 doses/day to about 12 doses/day, about 5 doses/day to about 6 doses/day, about 5 doses/day to about 7 doses/day, about 5 doses/day to about 8 doses/day, about 5 doses/day to about 9 doses/day, about 5 doses/day to about 10 doses/day, about 5 doses/day to about 11 doses/day, about 5 doses/day to about 12 doses/day, about 6 doses/day to about 7 doses/day, about 6 doses/day to about 8 doses/day, about 6 doses/day to about 9 doses/day, about 6 doses/day to about 10 doses/day, about 6 doses/day to about 11 doses/day, about 6 doses/day to about 12 doses/day, about 7 doses/day to about 8 doses/day, about 7 doses/day to about 9 doses/day, about 7 doses/day to about 10 doses/day, about 7 doses/day to about 11 doses/day, about 7 doses/day to about 12 doses/day, about 8 doses/day to about 9 doses/day, about 8 doses/day to about 10 doses/day, about 8 doses/day to about 11 doses/day, about 8 doses/day to about 12 doses/day, about 9 doses/day to about 10 doses/day, about 9 doses/day to about 11 doses/day, about 9 doses/day to about 12 doses/day, about 10 doses/day to about 11 doses/day, about 10 doses/day to about 12 doses/day, or about 11 doses/day to about 12 doses/day. In some embodiments, a pharmaceutical composition is administered in about 1 doses/day, about 2 doses/day, about 3 doses/day, about 4 doses/day, about 5 doses/day, about 6 doses/day, about 7 doses/day, about 8 doses/day, about 9 doses/day, about 10 doses/day, about 11 doses/day, or about 12 doses/day. In some embodiments, a pharmaceutical composition is administered in at least about 1 doses/day, about 2 doses/day, about 3 doses/day, about 4 doses/day, about 5 doses/day, about 6 doses/day, about 7 doses/day, about 8 doses/day, about 9 doses/day, about 10 doses/day, or about 11 doses/day. In some embodiments, a pharmaceutical composition is administered in at most about 2 doses/day, about 3 doses/day, about 4 doses/day, about 5 doses/day, about 6 doses/day, about 7 doses/day, about 8 doses/day, about 9 doses/day, about 10 doses/day, about 11 doses/day, or about 12 doses/day.

In some embodiments, the first agent is administered in a dose of about 1 mg to about 1,000 mg. In some embodiments, the first agent is administered in a dose of about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 1 mg to about 10 mg, about 1 mg to about 20 mg, about 1 mg to about 50 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 2 mg to about 20 mg, about 2 mg to about 50 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 3 mg to about 20 mg, about 3 mg to about 50 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 4 mg to about 20 mg, about 4 mg to about 50 mg, about 5 mg to about 6 mg, about 5 mg to about 7 mg, about 5 mg to about 8 mg, about 5 mg to about 9 mg, about 5 mg to about 10 mg, about 5 mg to about 20 mg, about 5 mg to about 50 mg, about 6 mg to about 7 mg, about 6 mg to about 8 mg, about 6 mg to about 9 mg, about 6 mg to about 10 mg, about 6 mg to about 20 mg, about 6 mg to about 50 mg, about 7 mg to about 8 mg, about 7 mg to about 9 mg, about 7 mg to about 10 mg, about 7 mg to about 20 mg, about 7 mg to about 50 mg, about 8 mg to about 9 mg, about 8 mg to about 10 mg, about 8 mg to about 20 mg, about 8 mg to about 50 mg, about 9 mg to about 10 mg, about 9 mg to about 20 mg, about 9 mg to about 50 mg, about 10 mg to about 20 mg, about 10 mg to about 50 mg, or about 20 mg to about 50 mg. In some embodiments, the first agent is administered in a dose of about 1 mg, about 2 mg, about 3 mg, about 4 mg, about 5 mg, about 6 mg, about 7 mg, about 8 mg, about 9 mg, about 10 mg, about 20 mg, or about 50 mg. In some embodiments, the first agent is administered in a dose of at least about 1 mg, about 2 mg, about 3 mg, about 4 mg, about 5 mg, about 6 mg, about 7 mg, about 8 mg, about 9 mg, about 10 mg, or about 20 mg. In some embodiments, the first agent is administered in a dose of at most about 2 mg, about 3 mg, about 4 mg, about 5 mg, about 6 mg, about 7 mg, about 8 mg, about 9 mg, about 10 mg, about 20 mg, or about 50 mg. In some embodiments, the first agent is administered in a dose of about 20 mg to about 250 mg. In some embodiments, the first agent is administered in a dose of about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 20 mg to about 150 mg, about 20 mg to about 200 mg, about 20 mg to about 250 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 30 mg to about 150 mg, about 30 mg to about 200 mg, about 30 mg to about 250 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 40 mg to about 150 mg, about 40 mg to about 200 mg, about 40 mg to about 250 mg, about 50 mg to about 60 mg, about 50 mg to about 70 mg, about 50 mg to about 80 mg, about 50 mg to about 90 mg, about 50 mg to about 100 mg, about 50 mg to about 150 mg, about 50 mg to about 200 mg, about 50 mg to about 250 mg, about 60 mg to about 70 mg, about 60 mg to about 80 mg, about 60 mg to about 90 mg, about 60 mg to about 100 mg, about 60 mg to about 150 mg, about 60 mg to about 200 mg, about 60 mg to about 250 mg, about 70 mg to about 80 mg, about 70 mg to about 90 mg, about 70 mg to about 100 mg, about 70 mg to about 150 mg, about 70 mg to about 200 mg, about 70 mg to about 250 mg, about 80 mg to about 90 mg, about 80 mg to about 100 mg, about 80 mg to about 150 mg, about 80 mg to about 200 mg, about 80 mg to about 250 mg, about 90 mg to about 100 mg, about 90 mg to about 150 mg, about 90 mg to about 200 mg, about 90 mg to about 250 mg, about 100 mg to about 150 mg, about 100 mg to about 200 mg, about 100 mg to about 250 mg, about 150 mg to about 200 mg, about 150 mg to about 250 mg, or about 200 mg to about 250 mg. In some embodiments, the first agent is administered in a dose of about 20 mg, about 30 mg, about 40 mg, about 50 mg, about 60 mg, about 70 mg, about 80 mg, about 90 mg, about 100 mg, about 150 mg, about 200 mg, or about 250 mg. In some embodiments, the first agent is administered in a dose of at least about 20 mg, about 30 mg, about 40 mg, about 50 mg, about 60 mg, about 70 mg, about 80 mg, about 90 mg, about 100 mg, about 150 mg, or about 200 mg. In some embodiments, the first agent is administered in a dose of at most about 30 mg, about 40 mg, about 50 mg, about 60 mg, about 70 mg, about 80 mg, about 90 mg, about 100 mg, about 150 mg, about 200 mg, or about 250 mg. In some embodiments, the first agent is administered in a dose of about 100 mg to about 1,000 mg. In some embodiments, the first agent is administered in a dose of about 100 mg to about 200 mg, about 100 mg to about 250 mg, about 100 mg to about 300 mg, about 100 mg to about 350 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1,000 mg, about 200 mg to about 250 mg, about 200 mg to about 300 mg, about 200 mg to about 350 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 250 mg to about 300 mg, about 250 mg to about 350 mg, about 250 mg to about 400 mg, about 250 mg to about 500 mg, about 250 mg to about 600 mg, about 250 mg to about 700 mg, about 250 mg to about 800 mg, about 250 mg to about 900 mg, about 250 mg to about 1,000 mg, about 300 mg to about 350 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 350 mg to about 400 mg, about 350 mg to about 500 mg, about 350 mg to about 600 mg, about 350 mg to about 700 mg, about 350 mg to about 800 mg, about 350 mg to about 900 mg, about 350 mg to about 1,000 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 500 mg to about 600 mg, about 500 mg to about 700 mg, about 500 mg to about 800 mg, about 500 mg to about 900 mg, about 500 mg to about 1,000 mg, about 600 mg to about 700 mg, about 600 mg to about 800 mg, about 600 mg to about 900 mg, about 600 mg to about 1,000 mg, about 700 mg to about 800 mg, about 700 mg to about 900 mg, about 700 mg to about 1,000 mg, about 800 mg to about 900 mg, about 800 mg to about 1,000 mg, or about 900 mg to about 1,000 mg. In some embodiments, the first agent is administered in a dose of about 100 mg, about 200 mg, about 250 mg, about 300 mg, about 350 mg, about 400 mg, about 500 mg, about 600 mg, about 700 mg, about 800 mg, about 900 mg, or about 1,000 mg. In some embodiments, the first agent is administered in a dose of at least about 100 mg, about 200 mg, about 250 mg, about 300 mg, about 350 mg, about 400 mg, about 500 mg, about 600 mg, about 700 mg, about 800 mg, or about 900 mg. In some embodiments, the first agent is administered in a dose of at most about 200 mg, about 250 mg, about 300 mg, about 350 mg, about 400 mg, about 500 mg, about 600 mg, about 700 mg, about 800 mg, about 900 mg, or about 1,000 mg. In some embodiments, the dose of the first agent is a therapeutically effective dose.

In some embodiments, the second agent is administered in a dose of about 1 mg to about 1000 mg. In some embodiments, the second agent is administered in a dose of about 1 mg to about 2 mg, about 1 mg to about 3 mg, about 1 mg to about 4 mg, about 1 mg to about 5 mg, about 1 mg to about 6 mg, about 1 mg to about 7 mg, about 1 mg to about 8 mg, about 1 mg to about 9 mg, about 1 mg to about 10 mg, about 1 mg to about 20 mg, about 1 mg to about 50 mg, about 2 mg to about 3 mg, about 2 mg to about 4 mg, about 2 mg to about 5 mg, about 2 mg to about 6 mg, about 2 mg to about 7 mg, about 2 mg to about 8 mg, about 2 mg to about 9 mg, about 2 mg to about 10 mg, about 2 mg to about 20 mg, about 2 mg to about 50 mg, about 3 mg to about 4 mg, about 3 mg to about 5 mg, about 3 mg to about 6 mg, about 3 mg to about 7 mg, about 3 mg to about 8 mg, about 3 mg to about 9 mg, about 3 mg to about 10 mg, about 3 mg to about 20 mg, about 3 mg to about 50 mg, about 4 mg to about 5 mg, about 4 mg to about 6 mg, about 4 mg to about 7 mg, about 4 mg to about 8 mg, about 4 mg to about 9 mg, about 4 mg to about 10 mg, about 4 mg to about 20 mg, about 4 mg to about 50 mg, about 5 mg to about 6 mg, about 5 mg to about 7 mg, about 5 mg to about 8 mg, about 5 mg to about 9 mg, about 5 mg to about 10 mg, about 5 mg to about 20 mg, about 5 mg to about 50 mg, about 6 mg to about 7 mg, about 6 mg to about 8 mg, about 6 mg to about 9 mg, about 6 mg to about 10 mg, about 6 mg to about 20 mg, about 6 mg to about 50 mg, about 7 mg to about 8 mg, about 7 mg to about 9 mg, about 7 mg to about 10 mg, about 7 mg to about 20 mg, about 7 mg to about 50 mg, about 8 mg to about 9 mg, about 8 mg to about 10 mg, about 8 mg to about 20 mg, about 8 mg to about 50 mg, about 9 mg to about 10 mg, about 9 mg to about 20 mg, about 9 mg to about 50 mg, about 10 mg to about 20 mg, about 10 mg to about 50 mg, or about 20 mg to about 50 mg. In some embodiments, the second agent is administered in a dose of about 1 mg, about 2 mg, about 3 mg, about 4 mg, about 5 mg, about 6 mg, about 7 mg, about 8 mg, about 9 mg, about 10 mg, about 20 mg, or about 50 mg. In some embodiments, the second agent is administered in a dose of at least about 1 mg, about 2 mg, about 3 mg, about 4 mg, about 5 mg, about 6 mg, about 7 mg, about 8 mg, about 9 mg, about 10 mg, or about 20 mg. In some embodiments, the second agent is administered in a dose of at most about 2 mg, about 3 mg, about 4 mg, about 5 mg, about 6 mg, about 7 mg, about 8 mg, about 9 mg, about 10 mg, about 20 mg, or about 50 mg. In some embodiments, the second agent is administered in a dose of about 20 mg to about 250 mg. In some embodiments, the second agent is administered in a dose of about 20 mg to about 30 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 20 mg to about 150 mg, about 20 mg to about 200 mg, about 20 mg to about 250 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 30 mg to about 150 mg, about 30 mg to about 200 mg, about 30 mg to about 250 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 40 mg to about 150 mg, about 40 mg to about 200 mg, about 40 mg to about 250 mg, about 50 mg to about 60 mg, about 50 mg to about 70 mg, about 50 mg to about 80 mg, about 50 mg to about 90 mg, about 50 mg to about 100 mg, about 50 mg to about 150 mg, about 50 mg to about 200 mg, about 50 mg to about 250 mg, about 60 mg to about 70 mg, about 60 mg to about 80 mg, about 60 mg to about 90 mg, about 60 mg to about 100 mg, about 60 mg to about 150 mg, about 60 mg to about 200 mg, about 60 mg to about 250 mg, about 70 mg to about 80 mg, about 70 mg to about 90 mg, about 70 mg to about 100 mg, about 70 mg to about 150 mg, about 70 mg to about 200 mg, about 70 mg to about 250 mg, about 80 mg to about 90 mg, about 80 mg to about 100 mg, about 80 mg to about 150 mg, about 80 mg to about 200 mg, about 80 mg to about 250 mg, about 90 mg to about 100 mg, about 90 mg to about 150 mg, about 90 mg to about 200 mg, about 90 mg to about 250 mg, about 100 mg to about 150 mg, about 100 mg to about 200 mg, about 100 mg to about 250 mg, about 150 mg to about 200 mg, about 150 mg to about 250 mg, or about 200 mg to about 250 mg. In some embodiments, the second agent is administered in a dose of about 20 mg, about 30 mg, about 40 mg, about 50 mg, about 60 mg, about 70 mg, about 80 mg, about 90 mg, about 100 mg, about 150 mg, about 200 mg, or about 250 mg. In some embodiments, the second agent is administered in a dose of at least about 20 mg, about 30 mg, about 40 mg, about 50 mg, about 60 mg, about 70 mg, about 80 mg, about 90 mg, about 100 mg, about 150 mg, or about 200 mg. In some embodiments, the second agent is administered in a dose of at most about 30 mg, about 40 mg, about 50 mg, about 60 mg, about 70 mg, about 80 mg, about 90 mg, about 100 mg, about 150 mg, about 200 mg, or about 250 mg. In some embodiments, the second agent is administered in a dose of about 100 mg to about 1,000 mg. In some embodiments, the second agent is administered in a dose of about 100 mg to about 200 mg, about 100 mg to about 250 mg, about 100 mg to about 300 mg, about 100 mg to about 350 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1,000 mg, about 200 mg to about 250 mg, about 200 mg to about 300 mg, about 200 mg to about 350 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 250 mg to about 300 mg, about 250 mg to about 350 mg, about 250 mg to about 400 mg, about 250 mg to about 500 mg, about 250 mg to about 600 mg, about 250 mg to about 700 mg, about 250 mg to about 800 mg, about 250 mg to about 900 mg, about 250 mg to about 1,000 mg, about 300 mg to about 350 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 350 mg to about 400 mg, about 350 mg to about 500 mg, about 350 mg to about 600 mg, about 350 mg to about 700 mg, about 350 mg to about 800 mg, about 350 mg to about 900 mg, about 350 mg to about 1,000 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 500 mg to about 600 mg, about 500 mg to about 700 mg, about 500 mg to about 800 mg, about 500 mg to about 900 mg, about 500 mg to about 1,000 mg, about 600 mg to about 700 mg, about 600 mg to about 800 mg, about 600 mg to about 900 mg, about 600 mg to about 1,000 mg, about 700 mg to about 800 mg, about 700 mg to about 900 mg, about 700 mg to about 1,000 mg, about 800 mg to about 900 mg, about 800 mg to about 1,000 mg, or about 900 mg to about 1,000 mg. In some embodiments, the second agent is administered in a dose of about 100 mg, about 200 mg, about 250 mg, about 300 mg, about 350 mg, about 400 mg, about 500 mg, about 600 mg, about 700 mg, about 800 mg, about 900 mg, or about 1,000 mg. In some embodiments, the second agent is administered in a dose of at least about 100 mg, about 200 mg, about 250 mg, about 300 mg, about 350 mg, about 400 mg, about 500 mg, about 600 mg, about 700 mg, about 800 mg, or about 900 mg. In some embodiments, the second agent is administered in a dose of at most about 200 mg, about 250 mg, about 300 mg, about 350 mg, about 400 mg, about 500 mg, about 600 mg, about 700 mg, about 800 mg, about 900 mg, or about 1,000 mg. In some embodiments, the dose of the second agent is a therapeutically effective dose.

In some embodiments, the ratio of the first agent to the second agent is about 0.01 to about 1. In some embodiments, the ratio of the first agent to the second agent is about 0.01 to about 0.05, about 0.01 to about 0.1, about 0.01 to about 0.2, about 0.01 to about 0.3, about 0.01 to about 0.4, about 0.01 to about 0.5, about 0.01 to about 0.6, about 0.01 to about 0.7, about 0.01 to about 0.8, about 0.01 to about 0.9, about 0.01 to about 1, about 0.05 to about 0.1, about 0.05 to about 0.2, about 0.05 to about 0.3, about 0.05 to about 0.4, about 0.05 to about 0.5, about 0.05 to about 0.6, about 0.05 to about 0.7, about 0.05 to about 0.8, about 0.05 to about 0.9, about 0.05 to about 1, about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.4 to about 0.5, about 0.4 to about 0.6, about 0.4 to about 0.7, about 0.4 to about 0.8, about 0.4 to about 0.9, about 0.4 to about 1, about 0.5 to about 0.6, about 0.5 to about 0.7, about 0.5 to about 0.8, about 0.5 to about 0.9, about 0.5 to about 1, about 0.6 to about 0.7, about 0.6 to about 0.8, about 0.6 to about 0.9, about 0.6 to about 1, about 0.7 to about 0.8, about 0.7 to about 0.9, about 0.7 to about 1, about 0.8 to about 0.9, about 0.8 to about 1, or about 0.9 to about 1. In some embodiments, the ratio of the first agent to the second agent is about 0.01, about 0.05, about 0.1, about 0.2, about 0.3, about 0.4, about 0.5, about 0.6, about 0.7, about 0.8, about 0.9, or about 1. In some embodiments, the ratio of the first agent to the second agent is at least about 0.01, about 0.05, about 0.1, about 0.2, about 0.3, about 0.4, about 0.5, about 0.6, about 0.7, about 0.8, or about 0.9. In some embodiments, the ratio of the first agent to the second agent is at most about 0.05, about 0.1, about 0.2, about 0.3, about 0.4, about 0.5, about 0.6, about 0.7, about 0.8, about 0.9, or about 1.

In some embodiments, the ratio of the second agent to the first agent is about 0.01 to about 1. In some embodiments, the ratio of the second agent to the first agent is about 0.01 to about 0.05, about 0.01 to about 0.1, about 0.01 to about 0.2, about 0.01 to about 0.3, about 0.01 to about 0.4, about 0.01 to about 0.5, about 0.01 to about 0.6, about 0.01 to about 0.7, about 0.01 to about 0.8, about 0.01 to about 0.9, about 0.01 to about 1, about 0.05 to about 0.1, about 0.05 to about 0.2, about 0.05 to about 0.3, about 0.05 to about 0.4, about 0.05 to about 0.5, about 0.05 to about 0.6, about 0.05 to about 0.7, about 0.05 to about 0.8, about 0.05 to about 0.9, about 0.05 to about 1, about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.4 to about 0.5, about 0.4 to about 0.6, about 0.4 to about 0.7, about 0.4 to about 0.8, about 0.4 to about 0.9, about 0.4 to about 1, about 0.5 to about 0.6, about 0.5 to about 0.7, about 0.5 to about 0.8, about 0.5 to about 0.9, about 0.5 to about 1, about 0.6 to about 0.7, about 0.6 to about 0.8, about 0.6 to about 0.9, about 0.6 to about 1, about 0.7 to about 0.8, about 0.7 to about 0.9, about 0.7 to about 1, about 0.8 to about 0.9, about 0.8 to about 1, or about 0.9 to about 1. In some embodiments, the ratio of the second agent to the first agent is about 0.01, about 0.05, about 0.1, about 0.2, about 0.3, about 0.4, about 0.5, about 0.6, about 0.7, about 0.8, about 0.9, or about 1. In some embodiments, the ratio of the second agent to the first agent is at least about 0.01, about 0.05, about 0.1, about 0.2, about 0.3, about 0.4, about 0.5, about 0.6, about 0.7, about 0.8, or about 0.9. In some embodiments, the ratio of the second agent to the first agent is at most about 0.05, about 0.1, about 0.2, about 0.3, about 0.4, about 0.5, about 0.6, about 0.7, about 0.8, about 0.9, or about 1.

In some embodiments, a minimum therapeutically effective dose of the first agent is reduced by about 5% when co-administered with the second agent compared to when administered alone to about 80% when co-administered with the second agent compared to when administered alone. In some embodiments, a minimum therapeutically effective dose of the first agent is reduced by about 5% when co-administered with the second agent compared to when administered alone to about 10% when co-administered with the second agent compared to when administered alone, about 5% when co-administered with the second agent compared to when administered alone to about 15% when co-administered with the second agent compared to when administered alone, about 5% when co-administered with the second agent compared to when administered alone to about 20% when co-administered with the second agent compared to when administered alone, about 5% when co-administered with the second agent compared to when administered alone to about 25% when co-administered with the second agent compared to when administered alone, about 5% when co-administered with the second agent compared to when administered alone to about 30% when co-administered with the second agent compared to when administered alone, about 5% when co-administered with the second agent compared to when administered alone to about 35% when co-administered with the second agent compared to when administered alone, about 5% when co-administered with the second agent compared to when administered alone to about 40% when co-administered with the second agent compared to when administered alone, about 5% when co-administered with the second agent compared to when administered alone to about 45% when co-administered with the second agent compared to when administered alone, about 5% when co-administered with the second agent compared to when administered alone to about 50% when co-administered with the second agent compared to when administered alone, about 5% when co-administered with the second agent compared to when administered alone to about 65% when co-administered with the second agent compared to when administered alone, about 5% when co-administered with the second agent compared to when administered alone to about 80% when co-administered with the second agent compared to when administered alone, about 10% when co-administered with the second agent compared to when administered alone to about 15% when co-administered with the second agent compared to when administered alone, about 10% when co-administered with the second agent compared to when administered alone to about 20% when co-administered with the second agent compared to when administered alone, about 10% when co-administered with the second agent compared to when administered alone to about 25% when co-administered with the second agent compared to when administered alone, about 10% when co-administered with the second agent compared to when administered alone to about 30% when co-administered with the second agent compared to when administered alone, about 10% when co-administered with the second agent compared to when administered alone to about 35% when co-administered with the second agent compared to when administered alone, about 10% when co-administered with the second agent compared to when administered alone to about 40% when co-administered with the second agent compared to when administered alone, about 10% when co-administered with the second agent compared to when administered alone to about 45% when co-administered with the second agent compared to when administered alone, about 10% when co-administered with the second agent compared to when administered alone to about 50% when co-administered with the second agent compared to when administered alone, about 10% when co-administered with the second agent compared to when administered alone to about 65% when co-administered with the second agent compared to when administered alone, about 10% when co-administered with the second agent compared to when administered alone to about 80% when co-administered with the second agent compared to when administered alone, about 15% when co-administered with the second agent compared to when administered alone to about 20% when co-administered with the second agent compared to when administered alone, about 15% when co-administered with the second agent compared to when administered alone to about 25% when co-administered with the second agent compared to when administered alone, about 15% when co-administered with the second agent compared to when administered alone to about 30% when co-administered with the second agent compared to when administered alone, about 15% when co-administered with the second agent compared to when administered alone to about 35% when co-administered with the second agent compared to when administered alone, about 15% when co-administered with the second agent compared to when administered alone to about 40% when co-administered with the second agent compared to when administered alone, about 15% when co-administered with the second agent compared to when administered alone to about 45% when co-administered with the second agent compared to when administered alone, about 15% when co-administered with the second agent compared to when administered alone to about 50% when co-administered with the second agent compared to when administered alone, about 15% when co-administered with the second agent compared to when administered alone to about 65% when co-administered with the second agent compared to when administered alone, about 15% when co-administered with the second agent compared to when administered alone to about 80% when co-administered with the second agent compared to when administered alone, about 20% when co-administered with the second agent compared to when administered alone to about 25% when co-administered with the second agent compared to when administered alone, about 20% when co-administered with the second agent compared to when administered alone to about 30% when co-administered with the second agent compared to when administered alone, about 20% when co-administered with the second agent compared to when administered alone to about 35% when co-administered with the second agent compared to when administered alone, about 20% when co-administered with the second agent compared to when administered alone to about 40% when co-administered with the second agent compared to when administered alone, about 20% when co-administered with the second agent compared to when administered alone to about 45% when co-administered with the second agent compared to when administered alone, about 20% when co-administered with the second agent compared to when administered alone to about 50% when co-administered with the second agent compared to when administered alone, about 20% when co-administered with the second agent compared to when administered alone to about 65% when co-administered with the second agent compared to when administered alone, about 20% when co-administered with the second agent compared to when administered alone to about 80% when co-administered with the second agent compared to when administered alone, about 25% when co-administered with the second agent compared to when administered alone to about 30% when co-administered with the second agent compared to when administered alone, about 25% when co-administered with the second agent compared to when administered alone to about 35% when co-administered with the second agent compared to when administered alone, about 25% when co-administered with the second agent compared to when administered alone to about 40% when co-administered with the second agent compared to when administered alone, about 25% when co-administered with the second agent compared to when administered alone to about 45% when co-administered with the second agent compared to when administered alone, about 25% when co-administered with the second agent compared to when administered alone to about 50% when co-administered with the second agent compared to when administered alone, about 25% when co-administered with the second agent compared to when administered alone to about 65% when co-administered with the second agent compared to when administered alone, about 25% when co-administered with the second agent compared to when administered alone to about 80% when co-administered with the second agent compared to when administered alone, about 30% when co-administered with the second agent compared to when administered alone to about 35% when co-administered with the second agent compared to when administered alone, about 30% when co-administered with the second agent compared to when administered alone to about 40% when co-administered with the second agent compared to when administered alone, about 30% when co-administered with the second agent compared to when administered alone to about 45% when co-administered with the second agent compared to when administered alone, about 30% when co-administered with the second agent compared to when administered alone to about 50% when co-administered with the second agent compared to when administered alone, about 30% when co-administered with the second agent compared to when administered alone to about 65% when co-administered with the second agent compared to when administered alone, about 30% when co-administered with the second agent compared to when administered alone to about 80% when co-administered with the second agent compared to when administered alone, about 35% when co-administered with the second agent compared to when administered alone to about 40% when co-administered with the second agent compared to when administered alone, about 35% when co-administered with the second agent compared to when administered alone to about 45% when co-administered with the second agent compared to when administered alone, about 35% when co-administered with the second agent compared to when administered alone to about 50% when co-administered with the second agent compared to when administered alone, about 35% when co-administered with the second agent compared to when administered alone to about 65% when co-administered with the second agent compared to when administered alone, about 35% when co-administered with the second agent compared to when administered alone to about 80% when co-administered with the second agent compared to when administered alone, about 40% when co-administered with the second agent compared to when administered alone to about 45% when co-administered with the second agent compared to when administered alone, about 40% when co-administered with the second agent compared to when administered alone to about 50% when co-administered with the second agent compared to when administered alone, about 40% when co-administered with the second agent compared to when administered alone to about 65% when co-administered with the second agent compared to when administered alone, about 40% when co-administered with the second agent compared to when administered alone to about 80% when co-administered with the second agent compared to when administered alone, about 45% when co-administered with the second agent compared to when administered alone to about 50% when co-administered with the second agent compared to when administered alone, about 45% when co-administered with the second agent compared to when administered alone to about 65% when co-administered with the second agent compared to when administered alone, about 45% when co-administered with the second agent compared to when administered alone to about 80% when co-administered with the second agent compared to when administered alone, about 50% when co-administered with the second agent compared to when administered alone to about 65% when co-administered with the second agent compared to when administered alone, about 50% when co-administered with the second agent compared to when administered alone to about 80% when co-administered with the second agent compared to when administered alone, or about 65% when co-administered with the second agent compared to when administered alone to about 80% when co-administered with the second agent compared to when administered alone. In some embodiments, a minimum therapeutically effective dose of the first agent is reduced by about 5% when co-administered with the second agent compared to when administered alone, about 10% when co-administered with the second agent compared to when administered alone, about 15% when co-administered with the second agent compared to when administered alone, about 20% when co-administered with the second agent compared to when administered alone, about 25% when co-administered with the second agent compared to when administered alone, about 30% when co-administered with the second agent compared to when administered alone, about 35% when co-administered with the second agent compared to when administered alone, about 40% when co-administered with the second agent compared to when administered alone, about 45% when co-administered with the second agent compared to when administered alone, about 50% when co-administered with the second agent compared to when administered alone, about 65% when co-administered with the second agent compared to when administered alone, or about 80% when co-administered with the second agent compared to when administered alone. In some embodiments, a minimum therapeutically effective dose of the first agent is reduced by at least about 5% when co-administered with the second agent compared to when administered alone, about 10% when co-administered with the second agent compared to when administered alone, about 15% when co-administered with the second agent compared to when administered alone, about 20% when co-administered with the second agent compared to when administered alone, about 25% when co-administered with the second agent compared to when administered alone, about 30% when co-administered with the second agent compared to when administered alone, about 35% when co-administered with the second agent compared to when administered alone, about 40% when co-administered with the second agent compared to when administered alone, about 45% when co-administered with the second agent compared to when administered alone, about 50% when co-administered with the second agent compared to when administered alone, or about 65% when co-administered with the second agent compared to when administered alone. In some embodiments, a minimum therapeutically effective dose of the first agent is reduced by at most about 10% when co-administered with the second agent compared to when administered alone, about 15% when co-administered with the second agent compared to when administered alone, about 20% when co-administered with the second agent compared to when administered alone, about 25% when co-administered with the second agent compared to when administered alone, about 30% when co-administered with the second agent compared to when administered alone, about 35% when co-administered with the second agent compared to when administered alone, about 40% when co-administered with the second agent compared to when administered alone, about 45% when co-administered with the second agent compared to when administered alone, about 50% when co-administered with the second agent compared to when administered alone, about 65% when co-administered with the second agent compared to when administered alone, or about 80% when co-administered with the second agent compared to when administered alone.

In some embodiments, a minimum therapeutically effective dose of the second agent is reduced by about 5% when co-administered with the first agent compared to when administered alone to about 80% when co-administered with the first agent compared to when administered alone. In some embodiments, a minimum therapeutically effective dose of the second agent is reduced by about 5% when co-administered with the first agent compared to when administered alone to about 10% when co-administered with the first agent compared to when administered alone, about 5% when co-administered with the first agent compared to when administered alone to about 15% when co-administered with the first agent compared to when administered alone, about 5% when co-administered with the first agent compared to when administered alone to about 20% when co-administered with the first agent compared to when administered alone, about 5% when co-administered with the first agent compared to when administered alone to about 25% when co-administered with the first agent compared to when administered alone, about 5% when co-administered with the first agent compared to when administered alone to about 30% when co-administered with the first agent compared to when administered alone, about 5% when co-administered with the first agent compared to when administered alone to about 35% when co-administered with the first agent compared to when administered alone, about 5% when co-administered with the first agent compared to when administered alone to about 40% when co-administered with the first agent compared to when administered alone, about 5% when co-administered with the first agent compared to when administered alone to about 45% when co-administered with the first agent compared to when administered alone, about 5% when co-administered with the first agent compared to when administered alone to about 50% when co-administered with the first agent compared to when administered alone, about 5% when co-administered with the first agent compared to when administered alone to about 65% when co-administered with the first agent compared to when administered alone, about 5% when co-administered with the first agent compared to when administered alone to about 80% when co-administered with the first agent compared to when administered alone, about 10% when co-administered with the first agent compared to when administered alone to about 15% when co-administered with the first agent compared to when administered alone, about 10% when co-administered with the first agent compared to when administered alone to about 20% when co-administered with the first agent compared to when administered alone, about 10% when co-administered with the first agent compared to when administered alone to about 25% when co-administered with the first agent compared to when administered alone, about 10% when co-administered with the first agent compared to when administered alone to about 30% when co-administered with the first agent compared to when administered alone, about 10% when co-administered with the first agent compared to when administered alone to about 35% when co-administered with the first agent compared to when administered alone, about 10% when co-administered with the first agent compared to when administered alone to about 40% when co-administered with the first agent compared to when administered alone, about 10% when co-administered with the first agent compared to when administered alone to about 45% when co-administered with the first agent compared to when administered alone, about 10% when co-administered with the first agent compared to when administered alone to about 50% when co-administered with the first agent compared to when administered alone, about 10% when co-administered with the first agent compared to when administered alone to about 65% when co-administered with the first agent compared to when administered alone, about 10% when co-administered with the first agent compared to when administered alone to about 80% when co-administered with the first agent compared to when administered alone, about 15% when co-administered with the first agent compared to when administered alone to about 20% when co-administered with the first agent compared to when administered alone, about 15% when co-administered with the first agent compared to when administered alone to about 25% when co-administered with the first agent compared to when administered alone, about 15% when co-administered with the first agent compared to when administered alone to about 30% when co-administered with the first agent compared to when administered alone, about 15% when co-administered with the first agent compared to when administered alone to about 35% when co-administered with the first agent compared to when administered alone, about 15% when co-administered with the first agent compared to when administered alone to about 40% when co-administered with the first agent compared to when administered alone, about 15% when co-administered with the first agent compared to when administered alone to about 45% when co-administered with the first agent compared to when administered alone, about 15% when co-administered with the first agent compared to when administered alone to about 50% when co-administered with the first agent compared to when administered alone, about 15% when co-administered with the first agent compared to when administered alone to about 65% when co-administered with the first agent compared to when administered alone, about 15% when co-administered with the first agent compared to when administered alone to about 80% when co-administered with the first agent compared to when administered alone, about 20% when co-administered with the first agent compared to when administered alone to about 25% when co-administered with the first agent compared to when administered alone, about 20% when co-administered with the first agent compared to when administered alone to about 30% when co-administered with the first agent compared to when administered alone, about 20% when co-administered with the first agent compared to when administered alone to about 35% when co-administered with the first agent compared to when administered alone, about 20% when co-administered with the first agent compared to when administered alone to about 40% when co-administered with the first agent compared to when administered alone, about 20% when co-administered with the first agent compared to when administered alone to about 45% when co-administered with the first agent compared to when administered alone, about 20% when co-administered with the first agent compared to when administered alone to about 50% when co-administered with the first agent compared to when administered alone, about 20% when co-administered with the first agent compared to when administered alone to about 65% when co-administered with the first agent compared to when administered alone, about 20% when co-administered with the first agent compared to when administered alone to about 80% when co-administered with the first agent compared to when administered alone, about 25% when co-administered with the first agent compared to when administered alone to about 30% when co-administered with the first agent compared to when administered alone, about 25% when co-administered with the first agent compared to when administered alone to about 35% when co-administered with the first agent compared to when administered alone, about 25% when co-administered with the first agent compared to when administered alone to about 40% when co-administered with the first agent compared to when administered alone, about 25% when co-administered with the first agent compared to when administered alone to about 45% when co-administered with the first agent compared to when administered alone, about 25% when co-administered with the first agent compared to when administered alone to about 50% when co-administered with the first agent compared to when administered alone, about 25% when co-administered with the first agent compared to when administered alone to about 65% when co-administered with the first agent compared to when administered alone, about 25% when co-administered with the first agent compared to when administered alone to about 80% when co-administered with the first agent compared to when administered alone, about 30% when co-administered with the first agent compared to when administered alone to about 35% when co-administered with the first agent compared to when administered alone, about 30% when co-administered with the first agent compared to when administered alone to about 40% when co-administered with the first agent compared to when administered alone, about 30% when co-administered with the first agent compared to when administered alone to about 45% when co-administered with the first agent compared to when administered alone, about 30% when co-administered with the first agent compared to when administered alone to about 50% when co-administered with the first agent compared to when administered alone, about 30% when co-administered with the first agent compared to when administered alone to about 65% when co-administered with the first agent compared to when administered alone, about 30% when co-administered with the first agent compared to when administered alone to about 80% when co-administered with the first agent compared to when administered alone, about 35% when co-administered with the first agent compared to when administered alone to about 40% when co-administered with the first agent compared to when administered alone, about 35% when co-administered with the first agent compared to when administered alone to about 45% when co-administered with the first agent compared to when administered alone, about 35% when co-administered with the first agent compared to when administered alone to about 50% when co-administered with the first agent compared to when administered alone, about 35% when co-administered with the first agent compared to when administered alone to about 65% when co-administered with the first agent compared to when administered alone, about 35% when co-administered with the first agent compared to when administered alone to about 80% when co-administered with the first agent compared to when administered alone, about 40% when co-administered with the first agent compared to when administered alone to about 45% when co-administered with the first agent compared to when administered alone, about 40% when co-administered with the first agent compared to when administered alone to about 50% when co-administered with the first agent compared to when administered alone, about 40% when co-administered with the first agent compared to when administered alone to about 65% when co-administered with the first agent compared to when administered alone, about 40% when co-administered with the first agent compared to when administered alone to about 80% when co-administered with the first agent compared to when administered alone, about 45% when co-administered with the first agent compared to when administered alone to about 50% when co-administered with the first agent compared to when administered alone, about 45% when co-administered with the first agent compared to when administered alone to about 65% when co-administered with the first agent compared to when administered alone, about 45% when co-administered with the first agent compared to when administered alone to about 80% when co-administered with the first agent compared to when administered alone, about 50% when co-administered with the first agent compared to when administered alone to about 65% when co-administered with the first agent compared to when administered alone, about 50% when co-administered with the first agent compared to when administered alone to about 80% when co-administered with the first agent compared to when administered alone, or about 65% when co-administered with the first agent compared to when administered alone to about 80% when co-administered with the first agent compared to when administered alone. In some embodiments, a minimum therapeutically effective dose of the second agent is reduced by about 5% when co-administered with the first agent compared to when administered alone, about 10% when co-administered with the first agent compared to when administered alone, about 15% when co-administered with the first agent compared to when administered alone, about 20% when co-administered with the first agent compared to when administered alone, about 25% when co-administered with the first agent compared to when administered alone, about 30% when co-administered with the first agent compared to when administered alone, about 35% when co-administered with the first agent compared to when administered alone, about 40% when co-administered with the first agent compared to when administered alone, about 45% when co-administered with the first agent compared to when administered alone, about 50% when co-administered with the first agent compared to when administered alone, about 65% when co-administered with the first agent compared to when administered alone, or about 80% when co-administered with the first agent compared to when administered alone. In some embodiments, a minimum therapeutically effective dose of the second agent is reduced by at least about 5% when co-administered with the first agent compared to when administered alone, about 10% when co-administered with the first agent compared to when administered alone, about 15% when co-administered with the first agent compared to when administered alone, about 20% when co-administered with the first agent compared to when administered alone, about 25% when co-administered with the first agent compared to when administered alone, about 30% when co-administered with the first agent compared to when administered alone, about 35% when co-administered with the first agent compared to when administered alone, about 40% when co-administered with the first agent compared to when administered alone, about 45% when co-administered with the first agent compared to when administered alone, about 50% when co-administered with the first agent compared to when administered alone, or about 65% when co-administered with the first agent compared to when administered alone. In some embodiments, a minimum therapeutically effective dose of the second agent is reduced by at most about 10% when co-administered with the first agent compared to when administered alone, about 15% when co-administered with the first agent compared to when administered alone, about 20% when co-administered with the first agent compared to when administered alone, about 25% when co-administered with the first agent compared to when administered alone, about 30% when co-administered with the first agent compared to when administered alone, about 35% when co-administered with the first agent compared to when administered alone, about 40% when co-administered with the first agent compared to when administered alone, about 45% when co-administered with the first agent compared to when administered alone, about 50% when co-administered with the first agent compared to when administered alone, about 65% when co-administered with the first agent compared to when administered alone, or about 80% when co-administered with the first agent compared to when administered alone.

The pharmaceutical compositions of this disclosure can be formulated to include either or both of the first and second agent required to perform treatment of a subject by combination therapy according to methods described herein. In some embodiments, the pharmaceutical compositions can be administered simultaneously or sequentially. In some embodiments, the pharmaceutical compositions can be administered in separate formulations.

The therapeutically effective amount of a first or second agent or a salt thereof can be administered before occurrence of the hearing disorders, such as, tinnitus, or Meniere's disease. The therapeutically effective amount of a first or second agent or a salt thereof can be administered at least about 1 hour before occurrence of the hearing disorders, for example, at least about 1 hour, 2 hours, 3 hours, 4 hours, 5 hours, 6 hours, before occurrence of the hearing disorder. The therapeutically effective amount of a first or second agent or a salt thereof can be administered less than about 1 month before occurrence of the hearing loss, for example, less than about 1 hour, 2 hours, 3 hours, 4 hours, 5 hours, 6 hours, before occurrence of the hearing disorders. In some cases, the therapeutically effective amount of a first or second agent or a salt thereof is administered at least 6 hours before occurrence of the hearing disorder.

The therapeutically effective amount of a first and/or second agent or a salt thereof can be administered after or contemporaneously upon occurrence of the hearing disorders, such as, for tinnitus, or Meniere's disease. The therapeutically effective amount of a first and/or second agent or a salt thereof can be administered at least about 1 minute after occurrence of the hearing disorders, for example, at least about 1 minute, 2 minutes, 3 minutes, 4 minutes, 5 minutes, 6 minutes, 12 minutes, 24 minutes, 36 minutes, 48 minutes, 54 minutes, or 60 minutes after occurrence of the hearing disorder. The therapeutically effective amount of a first and/or second agent or a salt thereof can be administered at least about 1 hour after occurrence of the hearing disorders, for example, at least about 1 hour, 2 hours, 3 hours, 4 hours, 5 hours, 6 hours after occurrence of the hearing disorder. The therapeutically effective amount of a first and/or second or a salt thereof can be administered less than about 1 month after occurrence of the hearing loss, for example, less than about 1 hour, 2 hours, 3 hours, 4 hours, 5 hours, 6 hours after occurrence of the hearing disorders. In some cases, the therapeutically effective amount of a first and/or second agent or a salt thereof is administered at least 12 hours after occurrence of the hearing disorder.

In some embodiments, the first agent is nimodipine (NMDP) or a salt thereof and the second agent is tetrandrine (TET) or a salt thereof. In some embodiments, the first agent is nimodipine (NMDP) or a salt thereof and the second agent is carbamazepine (CBZ) or a salt thereof. In some embodiments, the first agent is amlodipine (AML) or a salt thereof and the second agent is carbamazepine or a salt thereof. In some embodiments, the first agent is nimodipine or a salt thereof and the second agent is amlodipine or a salt thereof. In some embodiments, the first agent is tetrandrine (TET) or a salt thereof and the second agent is nimodipine or a salt thereof. In some embodiments, the first agent is tetrandrine or a salt thereof and the second agent is carbamazepine or a salt thereof. In some embodiments, the first agent is tetrandrine or a salt thereof and the second agent is amlodipine or a salt thereof.

can be in a solution dosage form. The solution dosage form can have a unit volume of at least about 1 mL, e.g. at least about 1, 2, 3, 4, 5, 6, 7, 8, 9, 10, 20, 30, 40, 50, 60, 70, 80, 90, 100, 200, 300, 400, 500, 600, 700, 800, or 900 mL. The solution dosage form can have a unit volume of less than about 1000 mL, e.g. less than about 1, 2, 3, 4, 5, 6, 7, 8, 9, 10, 20, 30, 40, 50, 60, 70, 80, 90, 100, 200, 300, 400, 500, 600, 700, 800, or 900 mL. The solution dosage form can have a unit volume ranges from about 1 to 500 mL, e.g. ranges 1-500, 1-300, 1-100, 1-80, 1-60, 1-40, 1-20, 1-10, 1-5, 10-500, 10-300, 10-100, 10-80, 10-60, 10-40, 10-20, 20-500, 20-300, 20-100, 20-80, 20-60, 20-40, 40-500, 40-300, 40-100, 40-80, 40-60, 60-500, 60-300, 60-100, 60-80, 80-500, 80-300, 80-100, 100-500, 100-300, or 300-500 mL.

### Combination Treatment

The therapeutically effective amount of a first and/or second agent or a salt thereof can be the sole active pharmaceutical ingredients (API). Alternatively, the therapeutically effective amount of a first and/or second agent or a salt thereof can be used in combination with one or more additional API. In some embodiments, the first and/or second agents and the additional API can be administered simultaneously or sequentially. In some embodiments, the first and/or second agents and the additional API can be administered in separate formulations. Disclosed herein are pharmaceuticals, compositions, kits, and methods of preventing or treating a hearing disorder by administering a first and/or second agent or a salt thereof, e.g. therapeutically effective amount of a first and/or second agent or a salt thereof, and one or more active pharmaceutical ingredients (API), e.g. therapeutically effective amount of API.

The one or more API can comprise one or more antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), 5HT serotonin reuptake inhibitors (5TH SRI), zonisamide, gabapentin, cannabinoid, or any combinations thereof.

Also disclosed herein are pharmaceuticals, compositions, kits, and methods for treating or preventing hearing loss in a subject in need thereof comprising administering a therapeutically effective amount of a first and/or second agent or a salt thereof and one or more active pharmaceutical ingredients (API). The one or more API can be administered in therapeutically effective amount. The one or more API can comprise one or more antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), 5HT serotonin reuptake inhibitors (5TH SRI), zonisamide, gabapentin, cannabinoid, or any combinations thereof.

The one or more API can comprise one or more antioxidants or spin-trapping agents. For example, the one or more antioxidants or spin-trapping agents can comprise allopurinol, glutathione, L-carnitine, methionine, or any combinations thereof. The one or more API can comprise one or more NMDA antagonists. For example, the one or more NMDA antagonists can comprise riluzole, caroverine, memantine, magnesium, or any combinations thereof. The one or more API can comprise one or more SSRI antagonists. For example, the one or more SSRI antagonists can comprise fluoxetine, sertraline, S-citalopram, alaproclate, or any combinations thereof. The one or more API can comprise one or more DRA. For example, the one or more DRA can comprise amantadine. The one or more API can comprise one or more acetylcholine release inducers or NERI. For example, the one or more acetylcholine release inducers or NERI can comprise bifemelane. The one or more API can comprise one or more MAI or SRI. For example, the one or more MAI or SRI can comprise pirlindole. The one or more API can comprise one or more SNRI. For example, the one or more SNRI can comprise milnacipran, bicifadine, or both. The one or more API can comprise one or more CCB. For example, the one or more CCB can comprise a first and/or second agent, verapamil, or both. The one or more API can comprise one or more NSRI. For example, the one or more NSRI can comprise atomoxetine. The one or more API can comprise one or more 5TH SRI. For example, the one or more 5TH SRI can comprise indeloxazine. The one or more API can comprise zonisamide.

The one or more API can comprise one or more drugs such as gabapentin. Other drugs that can be used are anticonvulsants. Other drugs can be used are drugs that stimulate gamma-aminobutyric acid (GABA) receptors. The one or more API can comprise a cannabinoid. For example, a cannabinoid can be marijuana or any extract of marijuana or synthetic composition that can stimulate the cannabinoid receptor, CB1 receptor, CB2 receptor, or a G-coupled receptor. These drugs can be used in in combination with nimodpine or a salt thereof.

Kits Disclosed herein are kits for treating or preventing hearing disorders in a subject in need thereof comprising a first and/or second agent or a salt thereof and a written instruction for treating or preventing hearing disorders using a first and/or second agent or a salt thereof. The a first and/or second agent or a salt thereof can be a therapeutically effective amount of a first and/or second agent or a salt thereof. The therapeutically effective amount of a first and/or second agent or a salt thereof can be in a tablet, capsule, caplet, spray, powder, gel cap, powder, or solution dosage form. For example, the therapeutically effective amount of a first and/or second agent or a salt thereof can be in a powder dosage form. The kit may further comprise a sterile solution. The kit may further comprise a sterile solution to be mixed with the powder dosage form prior to administration of the therapeutically effective amount of a first and/or second agent or a salt thereof. The kit may further comprise one or more active pharmaceutical ingredients (API). The one or more API can comprise one or more antioxidants, spin-trapping agents, N-methyl-D-aspartate (NMDA) antagonists, selective serotonin reuptake inhibitor (SSRI) antagonists, dopamine releasing agents (DRA), acetylcholine release inducers, norepinephrine reuptake inhibitors (NERI), monamineoxidase-A inhibitors (MAI), serotonin reuptake inhibitors (SRI), serotonin-norepinephrine reuptake inhibitors (SNRI), norepinephrine selective reuptake inhibitors (NSRI), 5HT serotonin reuptake inhibitors (5TH SRI), zonisamide, gabapentin, cannabinoid, or any combinations thereof.

### Patient Selection

In one aspect, presented herein a method of selecting a subject in need thereof for treatment of hearing disorder or a symptom of hearing disorder comprising: administering an L-type calcium channel blocker to the subject; selecting the patient for treatment of hearing disorder or a symptom of hearing disorder if the subject was responsive to the L-type calcium channel blocker; and administering a therapeutically effective amount of a first and/or second agent or a salt thereof to the subject if the subject was responsive to the L-type calcium channel blocker. In some cases, the L-type calcium channel blocker comprises carbamazepine. In some cases, the patient exhibits hearing loss. In some cases, the patient exhibits one or more symptoms related to a) hearing loss b) a change in auditory speech recognition as measured by a words-in-noise test, c) a change in auditory speech recognition as measured by a digits-in-noise test, d) a change in low-frequency hearing threshold e) a change in tinnitus severity g) a change in tinnitus loudness h) a change in vertigo severity i) a change in aural fullness j) a change in dizziness, and k) a change in hair cell function as observed when measured by a change in ABR threshold.

In one aspect, presented herein a method of selecting a subject in need thereof for treatment of tinnitus, or Meniere's disease or a symptom of tinnitus, or Meniere's disease comprising: administering an L-type calcium channel blocker to the subject; selecting the patient for treatment of tinnitus or a symptom of tinnitus if the subject was responsive to the L-type calcium channel blocker; and administering a therapeutically effective amount of a first and/or second agent or a salt thereof to the subject if the subject was responsive to the L-type calcium channel blocker. In some cases, the L-type calcium channel blocker comprises carbamazepine.

### Conditions to Be Treated

The hearing disorders in the subject in need thereof can be NIHL, ARHL, hearing loss due to drugs or injury, or tinnitus. The hearing disorder in the subject can also include a combination of two or all four listed diseases. For example, both tinnitus, or Meniere's disease and the drug-induced hearing loss can be caused by an ototoxic drug. The ototoxic drug can comprise a chemotherapeutic agent, an antineoplastic agent, an antibiotic, a loop-diuretic, a quinine or quinine-like compound, or a salicylate or salicylate-like compound. In some cases, the ototoxic drug is not streptomycin. In some cases, the antibiotic is not streptomycin. In some cases, the hearing disorder is not caused by streptomycin.

Disclosed herein are pharmaceuticals, compositions, kits, and methods for the prevention and/or treatment of hearing disorders, including, but not limited to, various conditions such as noise-induced hearing loss (NIHL), age-related hearing loss (ARHL or presbycusis), drug or injury-induced hearing loss, central auditory hearing disorder (CAPD), tinnitus, or Meniere's disease.

### NIHL—Noise-Induced Hearing Loss

NIHL is one of the most predominant health hazards posed by occupational and recreational settings. However, there are currently no FDA-approved drugs in diminishing NIHL, and the development of an efficacious treatment has been hampered by the complex array of cellular and molecular pathways involved in NIHL. NIHL can cause damages range from exhaustion of the hair cells in the ear to loss of those cells. Therefore, NIHL can be the consequence of overstimulation of the hair cells and supporting structures. Structural damage to hair cells (primarily the outer hair cells) can result in hearing loss that can be characterized by an attenuation and distortion of incoming auditory stimuli.

NIHL can be caused by a one-time exposure to excessive noise. For example, exposure to sound in excess of 80 dB, 90 dB, 100 dB, 110 dB, 120 dB, 130 dB, 140 dB, or 150 dB in short periods can cause NIHL. Alternatively, NIHL can also be caused by repeated exposure to noise over a period of time. For example, exposure to sound in excess of 60 dB, 65 dB, 70 dB, 75 dB, 80 dB, 85 dB, 90 dB, 95 dB, or 100 dB for more than 8 hours per day can cause NIHL. The symptoms of NIHL may include tinnitus, ear pain, hyperacusis, dizziness, vertigo and/or vestibular damages in the inner-ear.

Disclosed herein are pharmaceuticals, compositions, kits, and methods of treating or preventing hearing loss (e.g., NIHL) in a subject in need thereof wherein the methods comprise administering a therapeutically effective amount of a first and/or second agent or a salt thereof. In some cases, the therapeutically effective amount of a first and/or second agent or a salt thereof is an amount sufficient to reduce, or eliminate the NIHL. In some cases, the therapeutically effective amount of a first and/or second agent or a salt thereof is an amount sufficient to halt or prevent the NIHL. In some cases, the therapeutically effective amount of a first and/or second agent or a salt thereof is an amount sufficient to reverse the NIHL or at least partially restore hearing. The subject can be at risk of developing hearing loss (e.g., NIHL). Alternatively, the subject can be suffering hearing loss (e.g., NIHL).

### ARHL or Presbycusis

ARHL or presbycusis is a major health problem for which there are currently no treatments or preventatives. Age-related hearing loss develops gradually over time and in its early stages may be practically imperceptible to the affected individual. The cause of an ARHL or presbycusis is generally considered to be degeneration of the auditory nervous system, especially the auditory nerves in the ears. It is the most common form of hearing loss in persons over 55 years of age.

Early noise injury can be a cause of ARHL or presbycusis. The subject can be suffering or at risk of developing hearing loss (e.g., ARHL or presbycusis). For example, the subject can be suffering or at risk of developing hearing loss (e.g., ARHL or presbycusis) at least at 30 years, 35 years, 40 years, 45 years, 50 years, 55 years, 60 years, 65 years, 70 years, 75 years, 80 years, 85years, 90 years, 95 years, or 100 years of age. In some cases, the subject can be 50 years of age.

Disclosed herein are pharmaceuticals, compositions, kits, and methods of treating or preventing hearing loss (e.g., ARHL or presbycusis) in a subject in need thereof wherein the methods comprise administering a therapeutically effective amount of a first and/or second agent or a salt thereof. In some cases, the therapeutically effective amount of a first and/or second agent or a salt thereof is an amount sufficient to reduce, or eliminate the ARHL or presbycusis. In some cases, the therapeutically effective amount of a first and/or second agent or a salt thereof is an amount sufficient to halt or prevent the ARHL or presbycusis. In some cases, the therapeutically effective amount of a first and/or second agent or a salt thereof is an amount sufficient to reverse the ARHL or presbycusis or at least partially restore hearing. The subject can be at risk of developing hearing loss (e.g., ARHL or presbycusis). Alternatively, the subject can be suffering hearing loss (e.g., ARHL or presbycusis).

### Injury or Drug-induced Hearing Loss

Ototoxic drugs, such as chemotherapeutic agents, antineoplastic agents, antibiotics, loop-diuretics, quinines or a quinine-like compounds, and salicylate or salicylate-like compounds, can cause drug-induced hearing loss. For example, aminoglycosides are antibiotics that have been used for the treatment of Gram-negative bacterial infections and some aerobic Gram-positive bacterial infections. Despite their utility, however, they have serious side effects, including ototoxicity associated with the destruction of the sensory hair cells in organ of Corti of the cochlea of the inner ear. In addition, surgery near or on auditory nerves can cause hearing loss, which subsequently causes tinnitus.

Disclosed herein are pharmaceuticals, compositions, kits, and methods of treating or preventing hearing loss (e.g., injury or drug-induced hearing loss) in a subject in need thereof wherein the methods comprise administering a therapeutically effective amount of a first and/or second agent or a salt thereof. In some cases, the therapeutically effective amount of a first and/or second agent or a salt thereof is an amount sufficient to reduce, or eliminate this type of hearing loss. In some cases, the therapeutically effective amount of a first and/or second agent or a salt thereof is an amount sufficient to halt or prevent this type of hearing loss. In some cases, the therapeutically effective amount of a first and/or second agent or a salt thereof is an amount sufficient to not only prevent the drug-induced hearing loss or at least partially restore hearing. The subject can be at risk of developing hearing loss (e.g., drug-induced hearing loss). Alternatively, the subject can be suffering hearing loss (e.g., drug-induced hearing loss). In some cases, the therapeutically effective amount of a first and/or second agent or a salt thereof is an amount sufficient to not only prevent the drug-induced hearing loss, but works synergistically with cancer drugs to kill cancer cells

The therapeutically effective amount of a first and/or second agent or a salt thereof can prevent onset of an injury-or drug-induced hearing loss. For example, disclosed herein are methods for preventing a drug-induced hearing loss, comprising administering a therapeutically effective amount of a first and/or second agent or a salt thereof to a subject in need thereof prior to administering to the subject one or more ototoxic drugs for the treatment of a condition other than hearing loss. The administering a therapeutically effective amount of a first and/or second agent or a salt thereof can begin up to about 12 months (e.g., 1 day to 60 days) prior to the administration of the one or more ototoxic drugs. The administering a therapeutically effective amount of a first and/or second agent or a salt thereof can also begin on the same day as the administration of the one or more ototoxic drugs. In some cases, the administering a therapeutically effective amount of a first and/or second agent or a salt thereof can begin up to about 12 months, e.g. 1 hour, 6 hours, 12 hours, 24 hours, 2 days, 4 days, 6 days, 8 days, 10 days, 20 days, 1 month, 2 months, 4 months, 6 months, 8 months, 10 months, or 12 months prior to the administration of the one or more ototoxic drugs. In some cases, the administering a therapeutically effective amount of a first and/or second agent or a salt thereof can begin 1 day to 12 months, e.g. 1 day to 2 days, 2 days to 10 days, 10 days to 1 months, 1 months to 3 months, 3 months to 6 months, or 6 months to 12 months, prior to the administration of the one or more ototoxic drugs.

The therapeutically effective amount of a first and/or second agent or a salt thereof can treat or prevent drug-induced hearing loss, such as amelioration of drug-induced hearing loss, reduction or elimination of tinnitus, or Meniere's disease, partial or total rehabilitation of hearing, or prevention of further hearing loss arising out of ototoxic effects of the one or more ototoxic drugs. The methods disclosed herein provide for dosing of a pharmaceutical composition in response to a noted decrease in hearing function arising out of, or occurring during, dosing of one or more ototoxic drugs.

The administering a therapeutically effective amount of a first and/or second agent or a salt thereof can be continued for the duration of the one or more ototoxic drugs. The administering a therapeutically effective amount of a first and/or second agent or a salt thereof can stop on the same day as the cessation of the one or more ototoxic drugs. The administering a therapeutically effective amount of a first and/or second agent or a salt thereof can continue for at least about 1 day, e.g. for at least about 1 day, 2 days, 4 days, 6 days, 8 days, 10 days, 20 days, 1 month, 2 months, 4 months, 6 months, 8 months, 10 months, or 12 months after cessation of the one or more ototoxic drugs. In some cases, the administering a therapeutically effective amount of a first and/or second agent or a salt thereof can continue for at least about 1 day to 12 months, e.g. 1 day to 2 days, 2 days to 10 days, 10 days to 1 months, 1 months to 3 months, 3 months to 6 months, or 6 months to 12 months after cessation of the one or more ototoxic drugs.

Some examples of ototoxic drugs include certain antibacterial and antineoplastic drugs. For example, some ototoxic drugs are chemotherapeutic agents, e.g. antineoplastic agents, and antibiotics. Other possible candidates include loop-diuretics, quinines or a quinine-like compound, and salicylate or salicylate-like compounds. Thus, disclosed herein are pharmaceuticals, compositions, kits, and methods of treating or preventing hearing loss caused by an ototoxic drug, wherein the ototoxic drug can be an antineoplastic agent (e.g., ototoxic aminoglycoside antibiotic) such as cisplatin, an antibiotic such as an aminoglycoside, a loop-diuretic, a quinine, a quinine-like compound, a salicylate or salicylate-like compound.

Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. Thus, disclosed herein are pharmaceuticals, compositions, kits, and methods of treating or preventing drug-induced hearing loss comprising administering to a subject, who has been, is being or will be treated with one or more aminoglycosides, a therapeutically effective amount of a pharmaceutical composition of the disclosure. In some cases, the ototoxic aminoglycoside antibiotic is not streptomycin. In some cases, the hearing disorder is not caused by ototoxic aminoglycoside antibiotics. In some cases, the hearing disorder is not caused by streptomycin.

Hearing impairments induced by aminoglycosides can be prevented or reduced by the pharmaceuticals, compositions, kits, and methods disclosed herein. Although the aminoglycosides are particularly useful due to their rapid bactericidal action against infections of aminoglycoside-susceptible organisms, their use has heretofore been limited to more severe, complicated infections because of ototoxic and nephrotoxic side-effects. For this reason the aminoglycosides have been considered to have a low therapeutic/risk ratio compared to other antibiotics used systemically. Thus, disclosed herein are also improved methods of treatment of aminoglycoside-susceptible infections, comprising administering to a subject an anti-bacterially effective amount of an aminoglycoside and a pharmaceutical composition disclosed herein. It is to be recognized that recommended doses of aminoglycosides have been established; and the methods disclosed herein are effective when administering aminoglycosides in a range of about 100 to about 500%, in particular about 100 to about 250%, and more particularly about 100 to about 150% of the currently recommended doses, which are available in general in the product labeling and package inserts for the commercially available drug aminoglycoside drug products. The improved methods provide prophylaxis against aminoglycoside-induced hearing loss and/or tinnitus, thereby expanding the therapeutic index of the aminoglycoside drug.

The disclosed pharmaceutical compositions can be co-administered with one or more ototoxic drugs in the same dosage form. For example, an improved method is provided for treatment of infection of a subject by administration of an aminoglycoside antibiotic and a therapeutically effective amount of a pharmaceutical composition disclosed herein. Alternatively, the aminoglycoside antibiotic and the pharmaceutical composition disclosed herein can be administered to the subject in separate dosage forms.

The one or more ototoxic drugs can also be chemotherapeutic drugs for treatment of cancer in a subject. For example, an improved method is provided for treatment of cancer in a subject by administration of a chemotherapeutic drug (e.g. antineoplastic chemotherapeutic agent) and a therapeutically effective amount of a pharmaceutical composition disclosed herein.

Ototoxic antineoplastic chemotherapeutic agents include cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Thus, the methods disclosed herein can be used to treat ototoxicity (e.g. drug-induced hearing loss) in a subject, who will be, is being, or has been treated with an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

### Central Auditory Hearing Disorder (CAPD)

Central auditory processing disorders (CAPD) relate to difficulties in the perceptual processing of auditory information in the central nervous system (CNS). Tests for CAPD can include: auditory discrimination tests; auditory temporal processing and patterning tests; dichotic speech tests; monaural low-redundancy speech tests; binaural interaction tests; electroacoustic measures; and electrophysiological measures.

Disclosed herein are pharmaceuticals, compositions, kits, and methods of treating or preventing hearing loss (e.g., CAPD) in a subject in need thereof wherein the methods comprise administering a therapeutically effective amount of a first and/or second agent or a salt thereof. In some cases, the therapeutically effective amount of a first and/or second agent or a salt thereof is an amount sufficient to reduce, or eliminate the CAPD. In some cases, the therapeutically effective amount of a first and/or second agent or a salt thereof is an amount sufficient to halt or prevent the CAPD. In some cases, the therapeutically effective amount of a first and/or second agent or a salt thereof is an amount sufficient to reverse the CAPD or at least partially restore hearing. The subject can be at risk of developing hearing loss (e.g., CAPD). Alternatively, the subject can be suffering hearing loss (e.g., CAPD).

### Tinnitus

Tinnitus is the perception of sound in the ears even without external auditory stimulation. The most frequent manifestation of tinnitus is a ringing in the ears; however, tinnitus can also present as crickets, whooshing, pulsing, ocean waves, buzzing, even music, Tinnitus can be temporary, intermittent or even permanent; and its severity can range from a quiet background ringing to an overwhelming auditory sensation that drowns out external sources of sound Tinnitus can be caused by one or more factors, such as administration of, or exposure to, ototoxic substances (such as an aspirin overdose), exposure to a short burst of extreme noise (e.g. gunshot or explosion) or prolonged exposure to high decibel noise (such as aircraft engine noise, high decibel music concerts or high decibel headphone usage), or central auditory processing disorders as discussed herein.

Disclosed herein are pharmaceuticals, compositions, kits, and methods of treating or preventing hearing disorder (e.g., tinnitus) in a subject in need thereof wherein the methods comprise administering a therapeutically effective amount of a first and/or second agent or a salt thereof. In some cases, the therapeutically effective amount of a first and/or second agent or a salt thereof is an amount sufficient to reduce, or eliminate the hearing disorder (e.g., tinnitus). In some cases, the therapeutically effective amount of a first and/or second agent or a salt thereof is an amount sufficient to halt or prevent the hearing disorder (e.g., tinnitus), or treat a symptom thereof. In some cases, the therapeutically effective amount of a first and/or second agent or a salt thereof is an amount sufficient to reverse the hearing disorder (e.g., tinnitus) or at least partially restore hearing. The subject can be at risk of developing the hearing disorder (e.g., tinnitus). Alternatively, the subject can be suffering the hearing disorder (e.g., tinnitus).

Meniere's disease

Meniere's disease is a disorder of the inner ear that can lead to ringing in the ears (tinnitus), reduction or loss in hearing, vertigo and a feeling of fullness or congestion in the ear. Attacks of dizziness may come on suddenly or after a short period of tinnitus or muffled hearing. Some people will have single attacks of dizziness separated by long periods of time. Others may experience many attacks closer together over a number of days. Some people with Meniere's disease have vertigo so extreme that they lose their balance and fall. Meniere's disease is often a severe and debilitating condition for afflicted patients.

Meniere's disease can develop at any age, but it is more likely to happen to adults between 40 and 60 years of age. The National Institute on Deafness and Other Communication Disorders (NIDCD) estimates that there are approximately 615,000 individuals in the United States, currently diagnosed with Meniere's disease, with 45,500 cases newly diagnosed each year. Meniere's disease usually affects only one ear. Without being bound to a particular theory, the symptoms of Meniere's disease may be caused by the buildup of fluid in the compartments of the inner ear. There is no known cure for Meniere's disease. There remains a significant need for improved pharmaceutical compositions and methods of use for treatment of Meniere's disease.

### Salts, Stereoisomers, and Derivatives

Although described above with reference specific to compounds, one can also utilize stereoisomers, metabolites, derivates, and/or salts of the active compounds. Examples of therapeutically acceptable salts include, but are not limited to, mineral or organic acid salts of basic residues such as amines, and alkali or organic salts of acidic residues such as carboxylic acids. The therapeutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The therapeutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; example, non-aqueous media like ether, ethyl acetate, ethanol, isopropanol, or acetonitrile. Lists of suitable salts are found in Remington's Pharmaceutical Sciences, 17th ed. (Mack Publishing Company, Easton, Pa, 1985, p. 1418).

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

A metabolite of the above-mentioned compounds results from biochemical processes by which living cells interact with the active parent drug or other formulas or compounds in vivo. Metabolites include products or intermediates from any metabolic pathway.

### Measurement of the Effect of the Pharmaceutical Compositions

The composition is intended for the treatment, alleviation or lessening of acute or breakthrough tinnitus or a symptom of tinnitus, based on an assessment that comprises a) measurement of a change in ABR threshold, b) a change in auditory speech recognition as measured by a words-in-noise test, c) a change in auditory speech recognition as measured by a digits-in-noise test, d) a change in low-frequency hearing threshold e) a change in incidence of adverse events after administration of the pharmaceutical composition, f) a change in tinnitus severity g) a change in tinnitus loudness h) a change in vertigo severity i) a change in aural fullness j) a change in dizziness, and k) a change in hair cell function as observed when measured by a change in ABR threshold, after administration of the pharmaceutical composition.

In a further aspect, the composition, dosage unit, use and method of the disclosure is characterized by the effect of the treatment on the acute tinnitus or a symptom of tinnitus as measured as described herein. One way to record tinnitus or a symptom of tinnitus according to the disclosure comprises the measurement of onset of tinnitus or a symptom of tinnitus relief. Just before administration of the treatment the time is measured, e.g., by starting a stopwatch. When the subject is certain of feeling a meaningful tinnitus or a symptom of tinnitus relief the time is recorded, e.g., by stopping the stopwatch. The composition of the disclosure, upon administration, has a tinnitus or a symptom of tinnitus reduction score in the range of 2 to 7, such as 2, 3, 4, 5, 6, and 7, preferably such as 3, 4, 5, and 6, as measured by PID upon delivery of no more than two dosage units, preferably after delivery of one dosage unit.

At least 50% of subjects obtaining onset within 15 minutes after administration of treatment will be considered a success. Likewise the duration of effect may be measured as the difference between onset of effect and the time point where the subject declares the effect to cease or the time when the subject takes rescue medication, whatever comes first. Duration of tinnitus or a symptom of tinnitus relief of at least half an hour experienced by at least 50% of the subjects will be considered a success.

Another measurement is the Tinnitus or a symptom of tinnitus Intensity (TI) scored on an 11-point numeric rating scale (0=no tinnitus or a symptom of tinnitus, 10=unendurable tinnitus or a symptom of tinnitus). TIᵢ is the tinnitus or a symptom of tinnitus intensity at the time point Tᵢ. The TIᵢ is measured at one or more of the following time points (Tᵢ) before treatment (baseline), at the time of meaningful tinnitus or a symptom of tinnitus relief, every 15 minutes after administration of treatment for the two first hours, and every 30 minutes for the next two hours. A 40% decrease of mean TI within 15 minutes after treatment may be considered a success. Naturally other time points may and intervals may be selected.

TI₀ is the baseline tinnitus or a symptom of tinnitus intensity (scored on a scale as disclosed above) before administration of treatment (at time T₀). Tinnitus or a symptom of tinnitus Intensity Difference (TID) is the TI₀ compared to the tinnitus or a symptom of tinnitus intensity at time points after the administration of treatment (TIᵢ). A mean TID of 2 obtained within 15 minutes after administration will be considered a success.

A further measurement is the area under the TID curve or the Sum of Tinnitus or a symptom of tinnitus Intensity Difference (STID), TI being measured at the time points disclosed above. A mean 4-hour STID of 3 will be considered a success.

One method relates to a tinnitus or a symptom of tinnitus intensity scale as disclosed herein wherein tinnitus or a symptom of tinnitus relief is measured as a tinnitus or a symptom of tinnitus intensity difference (TID) of at least 30%, such as at least 40% based on a tinnitus or a symptom of tinnitus score measured close to the time of the administration TI₀ and a tinnitus or a symptom of tinnitus score measured at the time TIᵢ after administration. The time after administration may be selected from the time of one or more of the following times 3 minutes, 5 minutes, 7 minutes, 10 minutes, 15 minutes, 20 minutes, and 30 minutes upon administration. These times is used when the purpose of the measuring is to evaluate the immediate effect of the administration. If a measurement of the duration of treatment is desired, the tinnitus or a symptom of tinnitus relief is measured as a tinnitus or a symptom of tinnitus intensity difference (TID) based on a score measured immediately before the administration TI₀ and at the time TIᵢ after administration, the time after administration being selected from the time 45 minutes, 60 minutes, 75 minutes, 90 minutes, and 120 minutes upon administration. One alternative is to measure an effect from a given time after administration to a later time, and in this respect the desired time range is selected individually.

The tinnitus or a symptom of tinnitus relief score may be measured in accordance with the method disclosed herein or on a scale of 1-100% wherein 100% is a tinnitus or a symptom of tinnitus described by the patient as unbearable and 0% is no tinnitus or a symptom of tinnitus at all. It is preferred that the score is at least 30% from the start to the maximum relief effect is obtained.

A further measurement is as explained above the sum of tinnitus or a symptom of tinnitus intensity difference (STID) based on a score measured immediately before the administration PI₀ and at the time PIᵢ after administration, the time after administration being selected from the time any time as desired and includes the times as disclosed herein. In an embodiment, the sum of tinnitus or a symptom of tinnitus intensity difference is measured from at least 2 values measured during a period of at least 30 minutes, preferably at least during 45 minutes, preferable at least during 60 minutes such as during 90 minutes. Furthermore, the sum of tinnitus or a symptom of tinnitus intensity difference may be measured from at least 5 values such as at least from 7 values, preferable from at least 10 values such as from 11, 12 or 13 values.

### Exemplary Formulation

### Formulations

The compounds, or therapeutically acceptable salts thereof, or polymorphic variations thereof, can be formulated as pharmaceutical compositions. Such compositions can be administered orally, auricularly, e.g. intratympanically, buccally, intravenously, parenterally, by inhalation spray, rectally, intradermally, transdermally, pulmonary, nasally or topically in dosage unit formulations containing conventional nontoxic therapeutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrastromal injection, or infusion techniques. In an embodiment the composition is administered orally. In an embodiment the composition is administered auricularly, e.g. intratympanically.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N. Y. (1980).

The active compounds (or therapeutically acceptable salts thereof) may be administered per se or in the form of a pharmaceutical composition wherein the active compound(s) is in admixture or mixture with one or more therapeutically acceptable carriers, excipients or diluents. Pharmaceutical compositions may be formulated in conventional manner using one or more therapeutically acceptable carriers comprising excipients and auxiliaries which facilitate processing of the active compounds into preparations which can be used therapeutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Therapeutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicon dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being therapeutically formulated. The pharmaceutical compositions may take the form of, for example, tablets or capsules prepared by conventional means with therapeutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a therapeutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

In some cases, first agent and/or the second agent or salt thereof is formulated as the sole active pharmaceutical ingredient (API) in a dosage form. Such first agent and/or the second agent or salt thereof dosage form may be used alone or in combination therapy with one or more additional dosages containing one or more active pharmaceutical ingredients for prevention or treatment of hearing loss. In such cases, the daily dosage first agent and/or the second agent TET or salt thereof is conveniently provided in a single dosage form as described herein, or may be divided amongst two, three, four or more dosages.

### Pegylation

In alternate embodiments, a first and/or second agent salts together with other biologically active peptides and proteins, are conjugated to polyalkylene oxide polymers, particularly polyethylene glycols (PEG).

The section headings used herein are for organizational purposes only and are not to be construed as limiting the subject matter described.

### II. EXAMPLES

The following examples are included for illustrative purposes only and are not intended to limit the scope of the disclosure.

### Example 1: Single Agent Treatment of Tinnitus

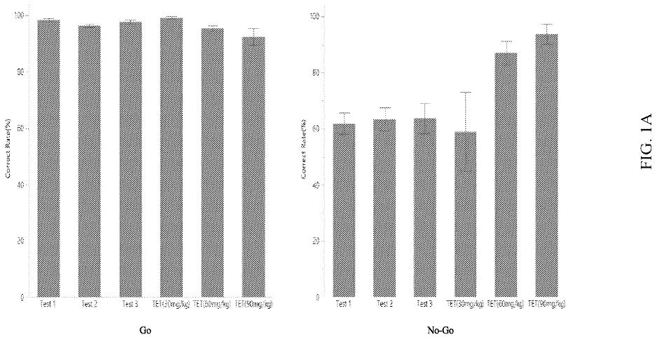

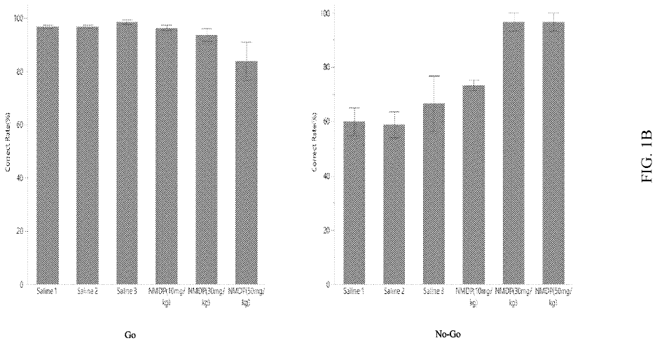

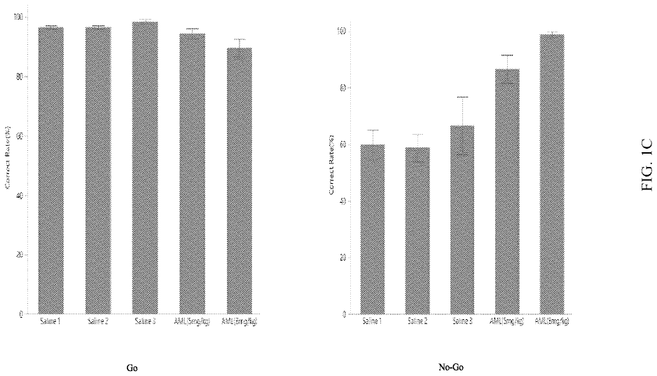

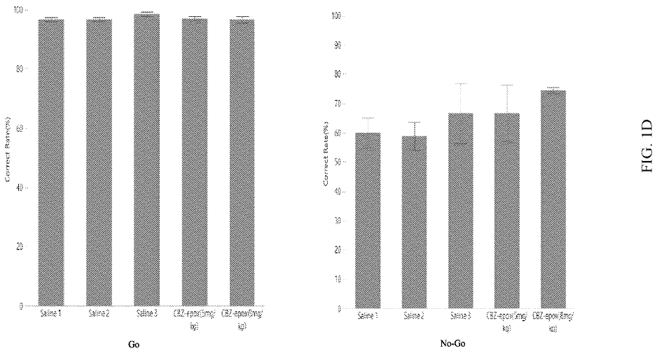

With reference to FIGS. 1A-1D, single drug treatment of tinnitus is evaluated using the go/no-go methodology further elaborated upon in Example 4.

FIG. 1A shows the results of the go/no-go trials for tinnitus positive mice treated with TET via intraperitoneal injection at dosages of 30, 60 and 90 mg/kg. All mice displayed a near 100% correct rate in the Go trial when un-dosed and when dosed at 30, 60 and 90 mg/kg of TET, indicating they were trained properly to perform the experiment. At a dosage of 30 mg/kg of TET in the no-go trial, no change in the correct percentage result is observed. At a dosage of 60 mg/kg of TET, about a 90% correct percentage result is observed, indicating that the 60 mg/kg of TET has a positive effect in treating the animal's tinnitus. Similarly, at a dosage of 90 mg/kg, about a 95% correct percentage result is observed, indicating that the 60 mg/kg of TET has a positive effect in treating the animal's tinnitus.

FIG. 1B shows the results of the go/no-go trials for tinnitus positive mice treated with NMDP via intraperitoneal injection at dosages of 10, 30, and 50 mg/kg. All mice displayed a near 100% correct rate in the Go trial when un-dosed and when dosed at 10, 30, and 50 mg/kg of NMDP, indicating they were trained properly to perform the experiment. At a dosage of 10 mg/kg of NMDP in the no-go trial, only a slight change in the correct percentage result is observed, within a margin of error. At a dosage of 30 mg/kg of NMDP, about a 95% correct percentage result is observed, indicating that the 30 mg/kg of NMDP has a positive effect in treating the animal's tinnitus. Similarly, at a dosage of 50 mg/kg, about a 95% correct percentage result is observed, indicating that the 50 mg/kg of NMDP has a positive effect in treating the animal's tinnitus.

FIG. 1C shows the results of the go/no-go trials for tinnitus positive mice treated with AML via intraperitoneal injection at dosages of 5, and 8 mg/kg. All mice displayed a near 100% correct rate in the Go trial when un-dosed and when dosed at 5, and 8 mg/kg of AML, indicating they were trained properly to perform the experiment. At a dosage of 5 mg/kg of AML, about a 90% correct percentage result is observed, indicating that the 5 mg/kg of AML has a positive effect in treating the animal's tinnitus. Similarly, at a dosage of 8 mg/kg, about a 95% correct percentage result is observed, indicating that the 8 mg/kg of AML has a positive effect in treating the animal's tinnitus.

FIG. 1D shows the results of the go/no-go trials for tinnitus positive mice treated with CBZ via intraperitoneal injection at dosages of 5, and 8 mg/kg. All mice displayed a near 100% correct rate in the Go trial when un-dosed and when dosed at 5, and 8 mg/kg of CBZ, indicating they were trained properly to perform the experiment. At a dosage of 5 mg/kg of CBZ, a less than 70% correct percentage result (within a margin of error of the saline dosed mice) is observed, indicating that the 5 mg/kg of CBZ has a minimal effect in treating the animal's tinnitus. Similarly, at a dosage of 8 mg/kg, a less than 80% correct percentage result is observed, indicating that the 8 mg/kg of CBZ has a minimal effect in treating the animal's tinnitus.

### Example 2: Combination Therapy for Treatment of Tinnitus

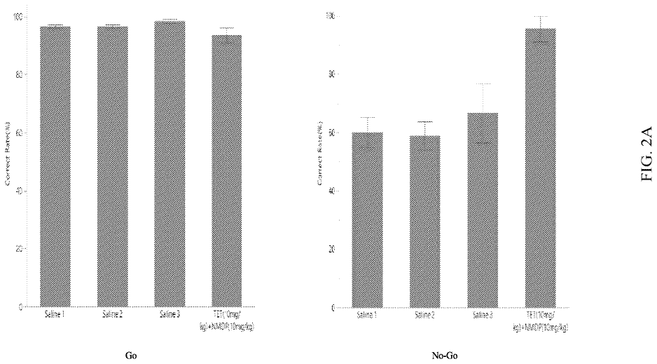

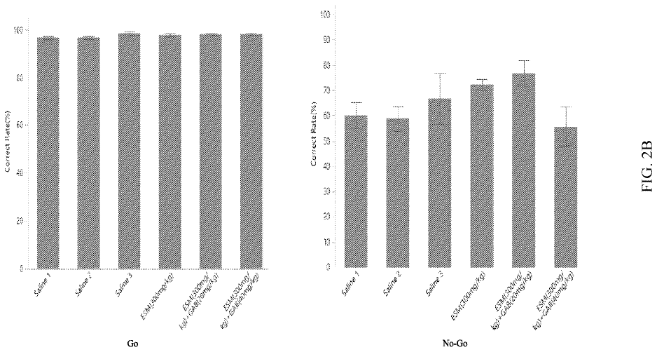

With reference to FIGS. 2A-2B, multi drug treatment of tinnitus is evaluated using the go/no-go methodology further elaborated upon in Example 4.

FIG. 2A shows the results of the go/no-go trials for tinnitus positive mice treated with TET and NMDP via intraperitoneal injection at dosages of 10 mg/kg of each agent. All mice displayed a near 100% correct rate in the Go trial when un-dosed and when dosed at 10 mg/kg of each agent, indicating they were trained properly to perform the experiment. At a dosage of 10 mg/kg of each agent, in the no-go trial, about a 95% correct percentage result is observed, indicating that the 60 mg/kg of TET has a positive effect in treating the animal's tinnitus. When compared to the results of TET alone (FIG. 1A) or NMDP alone (FIG. 1B), both of which were not effective in treating the animals tinnitus until they reached dosages of about 30 mg/kg; it is observed that the combination of TET and NMDP at dosages of 10 mg/kg displays a synergistic effect when combined, which is greater than the agents acting alone, or merely as an additive effect.. The results of FIG. 2A may indicate that the effect of TET shown in 2A may be due to blocking of L-type calcium channels as opposed to T-type calcium channels.

FIG. 2B shows the results of the go/no-go trials for tinnitus positive mice treated with ESM at 300 mg/kg, 300 mg/kg ESM and 20 mg/kg GAB, and at 300 mg/kg ESM and 40 mg/kg GAB via intraperitoneal injection. All mice displayed a near 100% correct rate in the Go trial when un-dosed and when dosed with ESM at 300 mg/kg, at 300 mg/kg ESM and 20 mg/kg GAB, and at 300 mg/kg ESM and 40 mg/kg GAB, indicating they were trained properly to perform the experiment. At a dosage of 300 mg/kg ESM, about a 70% correct percentage result is observed, indicating that the 300 mg/kg of ESM has a minimal effect in treating the animal's tinnitus. At a dosage of 300 mg/kg ESM and 20 mg/kg GAB, a slight increase in the correct percentage score up to about 80% correct is observed, indicating that the 300 mg/kg ESM and 20 mg/kg GAB had only a modest effect in treating the animal's tinnitus. At a dosage of 300 mg/kg ESM and 40 mg/kg GAB, a decrease in the correct percentage score is observed to about 55%, indicating that the 300 mg/kg ESM and 40 mg/kg GAB had no effect in treating the animal's tinnitus, or worsened the animal's tinnitus. FIG. 2B illustrates the unpredictable behavior of calcium channel inhibitors when acting in combination, and further illustrates that such combinations can have a negative or deleterious effect.

### Example 3 Treatment of Induced Tinnitus in Mice

In this example, mice suffering from noise induced tinnitus are treated with an intranasal delivery formulation of a first and/or second agent, e.g., the formulation of Example 2.

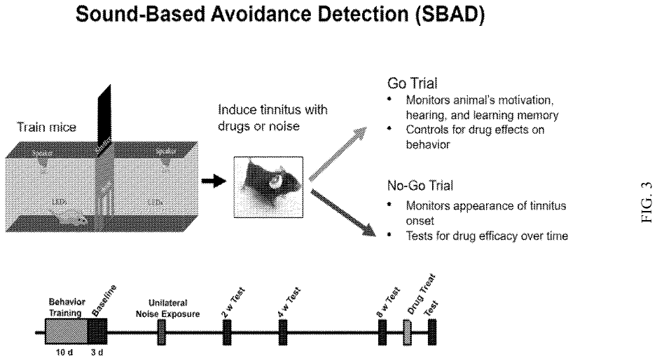

A sound-based avoidance detection (SBAD) method, as illustrated in FIG. 3, was used for detecting tinnitus and testing the animals'response to different pharmacological doses of a first and/or second agent. Using a shuttle box divided into two compartments, mice were trained to cross from side to side with sound cues in the “Go” trials and remain still in the absence of sound in the “No-Go” trials for 15 days. The animals had a 5 minute acclimation period in the shuttle box before testing began with 100 randomly assigned trials per day, lasting approximately 30-40 minutes. The sound cues were randomly played as white noise or narrow-band noise at 8,10,12.5,16, and 29 kHz with sound intensities randomly at 75, 80, or 85 dB. To reinforce the training sessions, the mice were shocked if they did not cross from one compartment to the other during the Go trials, and as well in the No-Go trials if they moved compartments in the absence of sound. Additionally, a hurdle was added on training 11 to reinforce the training. After the 15^{th} training day, mice were tested for 3 days to obtain a baseline score to be compared after noise exposure, as well as retraining for 3 days.

Following the successful completion of training so that mice reached a high percentage of success in the trials, the mice were exposed to a noise-induced trauma for tinnitus over a two day period. To ensure that the mice still have their hearing, one ear was protected with an ear plug, while the other ear was exposed to the noise. The mice were placed in a sound booth where they were exposed to 120 dB broadband noise at 4-25 kHz for two hours. Following the noise exposure, the mice were housed for one month, and then tested to see if they have developed tinnitus. In these testing periods, the mice were tested for three days in the shuttle boxes, where they underwent the Go and No-Go trials, however in the No-Go trials the mice were not shocked, as it is expected that mice with tinnitus would cross compartments in the absence of a sound cue. The mice were continued to be shocked in the Go trials in order to ensure that they have retained their hearing and can hear the sound cues. In examining the results of the tests following noise exposure, tinnitus positive mice had a higher average of errors in the No-Go trials in comparison to their training. To insure there is a significant difference in the No-Go trial scores, chi-square tests were completed comparing the post-noise exposure tests to the baseline tests. However, if tinnitus is not present in the mice, tests were done after waiting one more month, and another if needed.

Mice received dosages of a first and/or second agent at 50, 30, and 10 mg/kg. The Go and No-Go scores for the tests were compared using chi-square tests in Social Science Statistics and JASP. The chi-square tests were used first to determine whether mice were tinnitus positive by comparing the No-Go baseline scores pre-noise exposure to the No-Go post-noise exposure scores, with a significant difference (p-value<0.05) in at least 2 of the tests indicating a tinnitus positive mouse. Following this conclusion, the mice were tested accordingly to determine if their No-Go scores could be improved by different drug doses and combinations. In the Go scores, a first and/or second agent did not have an effect on the mice's ability to hear the sounds presented. In the No-Go trials, a first and/or second agent did not affect the mice's scores, indicating that the concentrations did not cause any behavioral changes and should be appropriate doses for testing.

The No-Go scores at 3 months post-noise exposure were compared to baselines to assess for tinnitus. Mice had significant decreases on all 3 days of testing. This indicates these mice had tinnitus. One or more mice is a control animal. In looking at the Go scores for the tinnitus animals when tested with the specified drugs, mice had significant decreases in their scores when. For mice administered a first and/or second agent, the No-Go scores were significantly increased. These results indicate a first and/or second agent is effective in treating this mouse's tinnitus. In contrast, a lack of change in the No-Go scores indicates the first and/or second agent was not effective in treating the animal's tinnitus, and decrease in the score may indicate that the animal's tinnitus was worsened.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for treating a hearing disorder or a symptom of a hearing disorder in a subject in need thereof, comprising the step of:
administering a therapeutically effective amount of a pharmaceutical composition to the subject,
wherein the pharmaceutical composition comprises
i) a first agent comprising a first L-type calcium channel blocker; and
ii) a second agent comprising one or more agents selected from the group consisting of calcium channel blockers, potassium channel openers, sodium channel blockers, gamma-aminobutyric acid type A (GABAA) receptor positive allosteric modulators, dopamine receptor antagonists, anticonvulsants, and serotonin antagonist and reuptake inhibitors (SARIs).

2. The method of claim 1, wherein the first agent comprises nimodipine (NMDP) or a salt thereof and wherein the second agent comprises one or more anticonvulsants selected from the group consisting of gabapentin and salts thereof, and carbamazepine (CBZ) and salts thereof.

3.-5. (canceled)

6. The method of claim 1, wherein the first agent comprises TET or a salt thereof and the second agent comprises CBZ or a salt thereof.

7.-14. (canceled)

15. A method of selecting a subject for treatment of a hearing disorder or a symptom of a hearing disorder, comprising the steps of:
a) administering to the subject a test agent comprising an L-type calcium channel blocker;
b) assessing whether the subject is responsive to the test agent; and
c) selecting the subject for administration of a pharmaceutical composition, if the subject is responsive to the test agent
wherein the pharmaceutical composition comprises:
i) a first agent comprising a first L-type calcium channel blocker; and
ii) a second agent comprising one or more agents selected from the group consisting of potassium channel openers, sodium channel blockers, gamma-aminobutyric acid type A (GABAA) receptor positive allosteric modulators, dopamine receptor antagonists, anticonvulsnts and serotonin antagonist and reuptake inhibiters (SARIs).
The subject is considered responsive to the test agent if one or more effects of the test agent are detected in the subject after administration of the test agent. The effects of the test agent include, but are not limited to a change of auditory and vestibular evoked potentials, a change of hearing thresholds, a change of tinnitus or vertigo testing scores, a change of life quality.

16. The method of claim 15, wherein the test agent is selected from the group consisting of tetrandrine (TET) and salts thereof, nimodipine (NMDP) and salts thereof, and carbamazepine (CBZ) and salts thereof.

17.-24. (canceled)

25. The method of claim 1, wherein the pharmaceutical composition is administered orally, nasally, intratympanically, buccally, intradermally, transdennally, topically, or via inhalation.

26.-28. (canceled)

29. The method of claim 28, wherein the pharmaceutical composition is administered by bringing the pharmaceutical composition in contact with a crista fenestrae cochlea, a round window, a tympanic cavity, a tympanic membrane, a auris media or an auris externa.

54. The method of claim 1, wherein the hearing disorder is selected from the group consisting of tinnitus, noise-induced hearing loss (NIHL), age-related hearing loss (ARHL), drug-induced hearing loss, or hearing loss due to cochlear pathology and Meniere's disease.

55.-61. (canceled)

62. The method of claim 15, wherein the assessing step comprises the step of:
measuring one or more effects of the test agent on the subject after administration of the test agent, wherein the one or more effects (including both increased and decreased changes) are selected from the group consisting of:
a) a change in auditory brainstem response (ABR) threshold,
b) a change in auditory speech recognition as measured by a words-in-noise test,
c) a change in auditory speech recognition as measured by a digits-in-noise test,
d) a change in low frequency hearing thresholds,
e) a change in incidence of adverse events after administration of the test agent,
f) a change in tinnitus severity,
g) a change in tinnitus loudness,
h) a change in vertigo severity,
i) a change in aural fullness,
j) a change in dizziness, and
k) a change in hair cell function.

63.-65. (canceled)

66. A pharmaceutical composition for treating a symptom of a hearing disorder, comprising:
a pharmaceutically effective amount of a first agent comprising an L-type calcium channel blocker; and
a pharmaceutically effective amount of a second agent comprising one or more agents selected from the group consisting of potassium channel openers, sodium channel blockers, gamma-aminobutyric acid type A (GABAA) receptor positive allosteric modulators, dopamine receptor antagonists, anticonvulsants and serotonin antagonist and reuptake inhibiters (SARIs).

67. (canceled)

68. The pharmaceutical composition of claim 66, wherein the first agent comprises TET or a salt thereof and wherein the second agent comprises carbamazepine (CBZ) or a salt thereof.

69.-70. (canceled)

71. The pharmaceutical composition of claim 66, wherein the first agent comprises NMDP or a salt thereof and wherein the second agent comprises CBZ or a salt thereof.

72.-106. (canceled)

107. The method of claim 15, further comprising the step of:
administering a therapeutically effective amount of the pharmaceutical composition to the subject.

108. The method of claim 107, where in the pharmaceutical composition comprises nimodipine (NMDP) and wherein the NMDP is administered at a daily range of 30-240 mg.
